(19) United States
(12) Patent Application Publication
(10) Pub. No.: US 2012/0142543 A1
(43) Pub. Date: Jun. 7, 2012

(54) Methods to assess treatment outcomes in Reward Deficiency Syndrome (RDS) behaviors utilizing expression profiling

(76) Inventor: Kenneth Blum, San Diego, CA (US)

(21) Appl. No.: 13/373,774

(22) Filed: Nov. 29, 2011

Related U.S. Application Data
(60) Provisional application No. 61/417,738, filed on Nov. 29, 2010.

(51) Int. Cl. C40B 30/00; C12Q 1/68; G01N 33/566

(52) U.S. Cl. 506/7; 435/7.1; 435/6.11

(57) ABSTRACT

The present invention relates to methods to objectively assess treatment outcomes in Reward Deficiency Syndrome (RDS) behaviors by obtaining expression profiles (e.g., mRNA expression and/or protein expression profiles) for one or more genes at two or more different time points, for example, before and after treating a subject known to have or suspected of having an RDS affliction. Analysis, for example, of mRNA and/or protein expression levels and/or patterns can be conducted before admission to a treatment facility, followed by testing at one or more various designated times during and after a subject's treatment. Such methods may also be combined with other tests, and can be used in diagnosis and treatment of RDS and RDS behaviors, including drug and/or alcohol abuse and addiction, overeating, gambling, sexual addiction, etc.

### RELATED APPLICATION

This application claims the benefit of and priority to U.S. provisional patent application Ser. No. 61/417,738, filed 29 Nov. 2010, the contents of which are hereby incorporated in their entirety for any and all purposes.

### BACKGROUND OF THE INVENTION

1. Field of the Invention

The present invention relates generally to objective methods for assessing the status of Reward Deficiency Syndrome (RDS) behaviors in subjects known to have or suspected of being afflicted with RDS.

2. Overview.

There exists great controversy regarding appropriate testing of gene polymorphisms and their role in disease and bodily function. As resources are limited, the debate revolves around whether enough progress has been made towards identifying the single nucleotide polymorphisms (SNPs) that are likely to contribute most to disease causation in order to justify investment in functional follow-up. Fortunately, nucleic acid sequencing and proteomics technologies are becoming less expensive and more accessible, allowing investigation of the causative role of strongest candidate SNPs available to date. What makes for strong candidates are significant disease associations with transcript expression and/or protein levels in various tissues.

Reward Deficiency Syndrome (RDS) results from a dysfunction in the Brain Reward Cascade that directly links abnormal craving behavior with a deficit in a number of reward genes, including dopaminergic, serotonergic, endorphinergic, catechoaminegic, gabaergic, adrenergic, opioidergic, and cholinergic genes, as well as many second messengers. As one example, dopamine is a very powerful neurotransmitter, which controls feelings of well-being. This sense of well-being is produced through the interaction of dopamine and neurotransmitters such as serotonin, the opioids (neuropeptides), and other powerful brain chemicals. For example, low serotonin has been associated with depression. High levels of opioids (the brain's opium) are associated with a sense of well-being.

3. Definitions.

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

Causal variant: In the context of GWAS it represents the SNP that is mechanistically linked to risk enhancement. This is distinct from SNPs that do not have any functional impact but are statistically associated with the disease phenotype because it is in linkage disequilibrium with the causal variant.

ChIP-Seq: Chromatin immunoprecipitation (ChIP) is a method to study protein-DNA interactions. It identifies genomic regions that are binding sites for a known protein. Analysis of these regions is typically performed by PCR, when there is a hypothesized known binding site, or through the use of genomic microarrays (ChIP-chip). Alternatively, analysis can be done using next-generation sequencing (Seq) technology to analyze DNA fragments.

CNV: Copy number variation is a type of structural variation in which a particular segment of the genome, typically larger than 1 kb, is found to have a variable copy number from a reference genome. Deep sequencing: a sequencing strategy used to reveal variations present at extremely low levels in a sample. For example, to identify rare somatic mutations found in a small number of cells in a tumor, or low abundance transcripts in transcriptome analysis.

DNA Methylation: A modification of the DNA that involves predominantly the addition of a methyl group to the 5 position of the pyrimidine ring of a cytosine found in a CpG dinucleotide sequence.

Epigenetic markers: an array of modifications to DNA and histones independent of changes in nucleotide sequence but rather the addition of methyl a methyl group to cytosine and a series of post-translation modifications of histone including methylation, acetylation, and phosphorylation.

Fine mapping: a strategy to identify other lower frequency variants in a disease-associated region (typically spanning a haplotype block) not represented in the initial genotyping platform with the goal of uncovering candidate causal variants. It can include data mining of publicly available sequencing efforts, such as the 1000 Genomes Project and targeted resequencing. Functional variant: a variant that confers a detectable functional impact on the locus. It can

represent a change in coding region but also changes in regulatory regions that have an impact on function.

GWAS: genome-wide association study is a case-control study design in which most loci in the genome are interrogated for association with a trait (disease) through the use of SNPs by comparing allele frequencies in cases and controls. Haplotype block: linear segments of the genome comprising coinherited alleles in the same chromosome.

Homologous recombination: an error-free recombination mechanism that exchanges genetic sequences between homologous loci during meiosis, and utilizes homologous sequences such as the sister-chromatid to promote DNA repair during mitosis.

Linkage disequilibrium: a nonrandom association between two markers (e.g. SNPs), which are typically close to one another due to reduced recombination between them. Supporting MicroRNAs: endogenous short (−23 nt) RNAs involved in gene regulation by pairing to mRNAs of protein coding mRNAs.

Next gen sequencing: a technology to sequence DNA in a massively parallel fashion, therefore sequencing is achieved at a much faster speed and lower cost than traditional methods.

Non-coding variant: a variant that is located outside of the coding region of a certain locus.

Tagging variant: a variant (SNP) that defines most of the haplotype diversity of a haplotype block.

Transcriptome: The complete set of transcripts in a cell. In some cases it can also include quantitative data about the amount of individual transcripts.

RNA-Seq: a method to obtain genome-wide transcription map using deep sequencing technologies to generate short sequence reads (30-400 bp). It reveals a transcriptional profile and levels of expression for each gene.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

A “plurality” means more than one.

The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

### SUMMARY OF THE INVENTION

The field is still making the first forays into the functional characterization of SNPs. Without wishing to be bound by theory, it is believed that causality can be inferred as being associated with a particular disease, condition, or affliction if a SNP leads to expression differences in reliable in vitro and/or in vivo assays. Thus, in the context of RDS behaviors, for example, a Substance Use Disorder (SUD) differential expression of one or more RDS behavior-associated genes (as analyzed, for example, by gene-based microarray analysis of isolated mRNA preparations and/or by analysis of the levels of proteins encoded by such genes) in response to various drugs of abuse or other addictive behaviors provides an avenue to objectively assess (on a qualitative, semi-quantitative, or quantitative basis) treatment outcomes, particularly for, for example, hypodopaminergic genes.

Thus, one aspect of the invention concerns methods of objectively assessing, qualitatively, semi-quantitatively, or quantitatively, a Reward Deficiency Syndrome (RDS) behavior in a subject known to have or suspected of having RDS. Such methods comprise obtaining a first expression profile (preferably of mRNA or protein) on a biological sample obtained from the subject at a first time point and a second expression profile on a biological sample obtained from the subject at a second time point, wherein the first and second expression profiles comprise measuring a level of an expression product, optionally a messenger RNA (mRNA) or a protein, for at least one gene selected from the group consisting of TrkB, Pomc, D4, prodynorphin (PDYN), Mu receptors, Kappa receptors, Dyn, Gpr88, Sgk, Cap1, PSD95, CamKII, DRD1A, Grm5, Adora2a, Homer1, Cnr1, Gpr6, hsp90beta, ProorphaninFQ/N, Orexin, cAMP-PKA, CART, micro-RNA miR-181a, NRXN3 beta, En1, D3 receptor, Preproenkephalin, mGluR8, GluR1, MOR, CREB phosphorylation, c fos, delta receptor, FTO, glucocorticoid receptor, G-alpha q-endogenous negative regulator of VMAT2, 5HT-2C, TH, alpha synuclein, intracellular JAK-STAT, Gsta4 (glutathione-S-transferase alpha 4), BDNF I, DeltaFosB, Dopamine D(2) receptor, tyrosine hydroxylase, alpha 6 subunit in catecholaminergic nuclei, c-jun, jun B, zif268, CCK, Neurotensin, dopamine reuptake transporter, COMT, MAO-A, Slc12a6, Dlgap2, Etnk1, Palm, Sqstm1, Nsg1, Akap9, Apba1, Stau1, Elavl4, Kif5a, Syt1, Hipk2, Araf, Cmip, NMDA, and NR1.

In preferred embodiments, the first expression profile is conducted prior to delivering a therapy to the subject intended to treat or alter the course of the Reward Deficiency Syndrome (RDS) behavior. In other embodiments, the second expression profile is conducted after delivering a therapy to the subject intended to treat or alter the course of the Reward Deficiency Syndrome (RDS) behavior. The biological samples are preferably derived from tissue samples obtained from the subject, wherein optionally the tissue samples are cell-containing samples optionally selected from the group consisting of blood, hair, mucous, saliva, and skin

In still other embodiments, the methods further include performing an allelic analysis on a biological sample from the subject to determine if the subject's genome contains at least one RDS-associated allele for each of two genes selected from the group consisting of DRD1, DRD2, DRD3, DRD4, DRD5, DAT1, PPARG, CHREBP, FTO, TNF-alpha, MANEA, Leptin OB, PEMT, MORA, MOAB, CRH, CRHEP, CRHR1, CRHR2, GAL, NPY, NPY1R, NPY2R, NPYY5R, ADIPOQ, STS, VDR, DBI, 5HTTIRP, GABRA2, GABRA3, GABBRA4, GABRA5, GABRB1, GABRB2, GABRB3, GABRD, GABRE, GARG2, GABRG2, GABRG3, GARBQ, SLC6A7, SLC6A11, SLC6A13, SLC32A1, GAD1, GAD2, DB1, MTHFR, VEGF, NOS3, HTR3B, SLC6A3, SLC6A4, COMT, DDC, OPRD1, OPRM1, OPRK1, ANKK1, HTR2A, HTR2C, HTRIA, HTR1B, HTR2A, HTR2B, HTR2C, HTR3A, HTR3B, ALDH1, ALDH2, CAT, CYP2E1, ADH1A, ALDH1B, ALDH1C, ADH4, ADH5, ADH6, ADH7, TPH1, TPH2, CNR1, CYP2E1, OPRKI, PDYN, PNOC, PRD1, OPRL1, PENK, POMC, GLA1, GLRA1, GLRB, GPHN, FAAH, CHRM1, CHRM2, CHRM3, CHRM4, CHRM5, CHRNA4, CHRNB2, ADRA1A, ADRA2B, ADRB2, SLC6A2, DRA2A, DRA2C, ARRB2, DBH, SCL18A2, TH, GR1K1, GRIN1, GRIN2A, GRIN2B, GRIN2C, GRM1, SLC6A4, ADCY7, AVPR1A, AVPRIB, CDK5RI, CREB1, CSNKIE, FEV, FOS, FOSL1, FOSL2, GSKK3B, JUN, MAPK1, MAPK3, MAPK14, MPD2, MGFB, NTRK2, NTSRI, NTSR2, PPP1R1B, PRKCE, BDNF, CART, CCK, CCKAR, CCKBR, CLOCK, HCRT, LEP, OXT, NR3C1, SLC29A1, and TAC1, wherein the allelic analysis is performed before, concurrently, or after the first expression profile; and, optionally determining a genetic addiction risk based on the results of the allelic analysis, wherein the genetic addiction risk takes into the account the presence of one or more of RDS-associated alleles among the genes analyzed, wherein the presence of at least one RDS-associated allele indicates a genetic addiction risk.

In still other preferred embodiments, the invention concerns methods wherein the RDS behavior is the subject's self-administration of a substance or activity of choice. For example, such substances or activities, and profiles to be assessed, include:
a. high fat food (HFF), wherein optionally the first and second expression profile experiments assess the mRNA of at least one gene selected from the group consisting of TrkB, Cart, Pomc, D2 receptor, D4 receptor, BDNF, Agrp, NPY, and Orexin receptor 2;
b. nor-binaltorphimine (opioid receptor antagonist), wherein optionally the first and second expression profile experiments assess the mRNA of at least one gene selected from the group consisting of PDYN and PENK;
c. housing and cognitive enrichment, wherein optionally the first and second expression profile experiments assess the mRNA of at least one gene selected from the group consisting of amygdala KOR and DOR opioid receptors and NPY5R;
d. morphine, wherein optionally the first and second expression profile experiments assess the mRNA of at least one gene selected from the group consisting of Mu receptors, Kappa receptors, PENK, PDYN, DYN, Gpr88, Sgk, Cap1, PSD95, CamKII, DRD1A, Grm5, Adora2a, Homer1, Cnr1, Gpr6, hsp90beta, ProorphaninFQ/N, POMC, CryB, CCK, Aq4, Gpr123, Gpr5 and Gal;
e. morphine withdrawal, wherein optionally the first and second expression profile experiments assess the mRNA of at least one gene selected from the group consisting of Mu receptors, POMC, orexin, PENK and Alpha-synuclein;
f. ethanol, wherein optionally the first and second expression profile experiments assess the mRNA of at least one gene selected from the group consisting of Mu receptors, PENK, POMC, PDYN, cAMP-PKA, CART, PNOC, OPRL-1, Drd2, all 8 GABA receptor subunits, 4 of 5 subunits of different glutamate receptors, and 7 enzymes involved with GABA and glutamate production (GAD-65, GAD-67, glutaminase, glutamate dehydrogenase, glutamine synthetase, aspartate aminotransferase (cytosolic and mitochondrial), cytochrome oxidase subunit III, Vlc, ATP synthase subunits A and C, Na K ATPase subunit alpha 1 and beta 1));
g. cocaine, wherein optionally the first and second expression profile experiments assess the mRNA of at least one gene selected from the group consisting of Mu receptors, PENK, PDYN, micro-RNA miR-181a, NRXN3 beta expression, CART, Ent, CD81, D3 receptor, Depamine receptors, ppDYN, DYN, Kappa Receptors, micro-RNAs miR-124, BDNF, D3R, orexin, Nurr1, Pitx3 and tyrosine hydroxylase;
h. cocaine withdrawal, wherein optionally the first and second expression profile experiments assess the mRNA of at least one gene selected from the group consisting of Mu receptors, PDYN, orexin, ppDYN and PENK;
i. Amphetamine, wherein optionally the first and second expression profile experiments assess the mRNA of at least one gene selected from the group consisting of PENK, PDYN, mGluR8, GluR1 and GluR2;
j. amphetamine withdrawal, wherein optionally the first and second expression profile experiments assess the mRNA of at least one gene selected from the group consisting of Mu receptors and PDYN;
k. Chronic nicotine treatment, wherein optionally the first and second expression profile experiments assess the mRNA of at least one gene selected from the group consisting of Mu receptors, POMC, PDYN, c-Fos, CREB phosphorylation, dopamine D2 receptor and tyrosine hydroxylase;
l. Alcohol cessation, wherein optionally the first and second expression profile experiments assess the mRNA of at least one gene selected from the group consisting of delta receptor;
m. Cannabinoid agonists (THC, CP-55,940 or R-methanandamide), wherein optionally the first and second expression profile experiments assess the mRNA of at least one gene selected from the group consisting of PENK and POMC;
n. cannabinoid withdrawal, wherein optionally the first and second expression profile experiments assess the mRNA of at least one gene selected from the group consisting of PENK;
o. Kappa receptor agonists (U-69593 or U-50,488H), wherein optionally the first and second expression profile experiments assess the mRNA of at least one gene selected from the group consisting of PDYN;
p. Methamphetamine, wherein optionally the first and second expression profile experiments assess the mRNA of at least one gene selected from the group consisting of PDYN and TNF-alpha;
q. food (effects on hypothalamic FTO), wherein optionally the first and second expression profile experiments assess the mRNA of at least one gene selected from the group consisting of FTO;
r. Leucine, wherein optionally the first and second expression profile experiments assess the mRNA of at least one gene selected from the group consisting of FTO;
s. dual orexin receptor antagonist (DORA)-antagonist of OX1R and OX2R, wherein optionally the first and second expression profile experiments assess the mRNA of at least one gene selected from the group consisting of
t. Aging, wherein optionally the first and second expression profile experiments assess the mRNA of at least one gene selected from the group consisting of orexin-receptor 2;
u. CREB, wherein optionally the first and second expression profile experiments assess the mRNA of at least one gene selected from the group consisting of
v. dopamine transporter (DAT—as influenced by overexpression or silencing in the nucleus accumbens), wherein optionally the first and second expression profile experiments assess the mRNA of at least one gene selected from the group consisting of
w. CREB, wherein optionally the first and second expression profile experiments assess the mRNA of at least one gene selected from the group consisting of CART;
x. deoxyribozyme 164 (DRz164)—cleaves Period 1 gene (Peri) mRNA. Injection with DRz164 before morphine treatment, wherein optionally the first and second expression profile experiments assess the mRNA of at least one gene selected from the group consisting of ERK and CREB;
y. para-chloroamphetamine (depletes 5-HT), wherein optionally the first and second expression profile experiments assess the mRNA of at least one gene selected from the group consisting of glucocorticoid receptor and BDNF;
z. predisposition for obesity (normal diet), wherein optionally the first and second expression profile experiments assess the mRNA of at least one gene selected from the group consisting of Galphaq, tyrosine hydroxylase, VMAT2, DAT, and D2S presynaptic autoreceptor;
aa. editing of serotonin 2C receptor mRNA (via ADAR enzyme), wherein optionally the first and second expression profile experiments assess the mRNA of at least one gene selected from the group consisting of 5HT-2C;
bb. Heroin, wherein optionally the first and second expression profile experiments assess the mRNA of at least one gene selected from the group consisting of PENK, D2 receptor, DAT, Nurr1 and tyrosine hydroxylase;
cc. social isolation, wherein optionally the first and second expression profile experiments assess the mRNA of at least one gene selected from the group consisting of D2 receptor;
dd. HSV vector mediated elevations in GluR1 or GluR2, wherein optionally the first and second expression profile experiments assess the mRNA of at least one gene selected from the group consisting of GluR1 and GluR2;
ee. high or low consumption of sugar, wherein optionally the first and second expression profile experiments assess the mRNA of at least one gene selected from the group consisting of 5HT2A, mGlu1, AMPA, GluR1, adrenergic alpha 2A, NMDA NR2B, GABA Alpha 3, adrenergic alpha2B, GluR2, GluR3, 5HT1B and GABA alpha5;
ff. Leptin receptor expression in VTA, wherein optionally the first and second expression profile experiments assess the mRNA of at least one gene selected from the group consisting of
gg. ethanol preference, wherein optionally the first and second expression profile experiments assess the mRNA of at least one gene selected from the group consisting of Gsta4, FAAH and CB1;
hh. morphine response (mice), wherein optionally the first and second expression profile experiments assess the mRNA of at least one gene selected from the group consisting of Atp I aw, COMT, Gabra I, GABA-A, Gabra2, Grm7, Kcnj 9, Syt4, Gfap, Mtap2, and Hprt I;
ii. psychostimulant (e.g. cocaine, amphetamine), wherein optionally the first and second expression profile experiments assess the mRNA of at least one gene selected from the group consisting of CART, cAMP and CREB;
jj. forskolin (intra-accumbal injection in rat), wherein optionally the first and second expression profile experiments assess the mRNA of at least one gene selected from the group consisting of CART;
kk. intrastriatal infusion of cholinergic muscarinic antagonist, wherein optionally the first and second expression profile experiments assess the mRNA of at least one gene selected from the group consisting of
ll. Delta-tetrahydrocannabinol, wherein optionally the first and second expression profile experiments assess the mRNA of at least one gene selected from the group consisting of BDNF, zif268 and MAPK/ERK;
mm. DeltaFosB, wherein optionally the first and second expression profile experiments assess the mRNA of at least one gene selected from the group consisting of
nn. Nandrolone decanoate, wherein optionally the first and second expression profile experiments assess the mRNA of at least one gene selected from the group consisting of D2 receptor and D1 receptor;
oo. Voluntary wheel running in addicted Lewis rats, wherein optionally the first and second expression profile experiments assess the mRNA of at least one gene selected from the group consisting of
pp. Substance P (during morphine withdrawal), wherein optionally the first and second expression profile experiments assess the mRNA of at least one gene selected from the group consisting of D2 receptor;
qq. U99194A (D(3) dopamine receptor antagonist), wherein optionally the first and second expression profile experiments assess the mRNA of at least one gene selected from the group consisting of c-Fos;
rr. cocaine, cocaine+nondrolone, or nandrolone alone, wherein optionally the first and second expression profile experiments assess the mRNA of at least one gene selected from the group consisting of
ss. Dextromethorphan, wherein optionally the first and second expression profile experiments assess the mRNA of at least one gene selected from the group consisting of tyrosine hydroxylase;
tt. Running, wherein optionally the first and second expression profile experiments assess the mRNA of at least one gene selected from the group consisting of DYN, GluR1, AMPA, NGFI-B and Nor1;
uu. Amitriptyline, wherein optionally the first and second expression profile experiments assess the mRNA of at least one gene selected from the group consisting of D1, D2 and D3 receptors;
vv. Desipramine, wherein optionally the first and second expression profile experiments assess the mRNA of at least one gene selected from the group consisting of D3 receptor;
ww. Imipramine, wherein optionally the first and second expression profile experiments assess the mRNA of at least one gene selected from the group consisting of D1, D2 and D3 receptors;
xx. Tranylcypromine, wherein optionally the first and second expression profile experiments assess the mRNA of at least one gene selected from the group consisting of D3 receptor;
yy. electroconvulsive therapy, wherein optionally the first and second expression profile experiments assess the mRNA of at least one gene selected from the group consisting of D3 receptor;
zz. Fetal alcohol syndrome, wherein optionally the first and second expression profile experiments assess the mRNA of at least one gene selected from the group consisting of c-fos, c-jun, jun B, zif268 and junB;
aaa. S(−)- and R (+)-salsolinol, wherein optionally the first and second expression profile experiments assess the mRNA of at least one gene selected from the group consisting of POMC and cAMP;
bbb. peripheral nerve injury (unilateral chronic constriction of sciatic nerve), wherein optionally the first and second expression profile experiments assess the mRNA of at least one gene selected from the group consisting of tyrosine hydroxylase and DRD2; and
ccc. alcohol and splice variants, wherein optionally the first and second expression profile experiments assess the mRNA of at least one gene selected from the group consisting of D2UD2S receptor ratio and NMDA NR1.

These and other aspects and embodiments of the invention are discussed in greater detail in the sections that follow.

### BRIEF DESCRIPTION OF THE FIGURES

This application contains at least one figure executed in color. Copies of this application with color drawing(s) are available upon request and payment of the necessary fee. A summary of each figure appears below.

FIG. 1: FIG. 1 (A) Schematic represents the normal physiologic state of the neurotransmitter interaction at the mesolimbic region of the brain. Briefly in terms of the “Brain Reward Cascade” first coined by Blum and Kozlowski [X]: serotonin in the hypothalamus stimulates neuronal projections of methionine enkephalin in the hypothalamus which in turn inhibits the release of GABA in the substania nigra thereby allowing for the normal amount of Dopamine to be released at the Nucleus Accumbens (reward site of Brain). (B) Represents hypodopaminergic function of the mesolimbic region of the brain. It is possible that the hypodopaminergic state is due to gene polymorphisms as well as environmental elements including both stress and neurotoxicity from aberrant abuse of psychoactive drugs (i.e. alcohol, heroin, cocaine etc). Genetic variables could include serotonergic genes (serotonergic receptors [5HT2a]; serotonin transporter 5HTIPR); endorphinergic genes (mu OPRM1 gene; proenkephalin (PENK) [PENK polymorphic 3′ UTR dinucleotide (CA) repeats}; GABergic gene (GABRB3) and dopaminergic genes (ANKKI Taq A; DRD2 C957T, DRD4 7R, COMT Val/met substation, MAO-A uVNTR, and SLC6A3 9 or 10R). Any of these genetic and or environmental impairments could result in reduced release of dopamine and or reduced number of dopaminergic receptors.

### DETAILED DESCRIPTION OF THE INVENTION

This invention concerns methods to assess biomarkers, particularly the level of gene products such as a messenger RNAs (mRNAs) and/or the proteins encoded by such mRNAs, common to overall wellness and, as such, attenuation of aberrant craving behaviors, including other detrimental behaviors in drug dependency. Particular emphasis is placed on individual drug or activity of choice. Such methods will benefit chemical dependency programs worldwide, as well as bariatric centers involved in the treatment of obesity or food cravings, as well as centers involved in gambling, internet, or sexual addiction, to name a few. This application is supported by a new definition of addiction as developed and release by American Society of Addiction Medicine (ASAM).

Short Definition of Addiction: Addiction is a primary, chronic disease of brain reward, motivation, memory, and related circuitry. Dysfunction in these circuits leads to characteristic biological, psychological, social, and spiritual manifestations. This is reflected in an individual pathologically pursuing reward and/or relief by substance use and other behaviors.

Addiction is characterized by inability to consistently abstain, impairment in behavioral control, craving, diminished recognition of significant problems with one's behaviors and interpersonal relationships, and a dysfunctional emotional response. Like other chronic diseases, addiction often involves cycles of relapse and remission. Without treatment or engagement in recovery activities, addiction is progressive and can result in disability or premature death.

Addiction affects neurotransmission and interactions within reward structures of the brain, including the nucleus accumbens, anterior cingulate cortex, basal forebrain and amygdala, such that motivational hierarchies are altered and addictive behaviors, which may or may not include alcohol and other drug use, supplant healthy, self-care related behaviors. Addiction also affects neurotransmission and interactions between cortical and hippocampal circuits and brain reward structures, such that the memory of previous exposures to rewards (such as food, sex, alcohol, and other drugs) leads to a biological and behavioral response to external cues, in turn triggering craving and/or engagement in addictive behaviors.

The neurobiology of addiction encompasses more than the neurochemistry of reward. The frontal cortex of the brain and underlying white matter connections between the frontal cortex and circuits of reward, motivation and memory are fundamental in the manifestations of altered impulse control, altered judgment, and the dysfunctional pursuit of rewards (which is often experienced by the affected person as a desire to “be normal”) seen in addiction—despite cumulative adverse consequences experienced from engagement in substance use and other addictive behaviors. The frontal lobes are important in inhibiting impulsivity and in assisting individuals to appropriately delay gratification. When persons with addiction manifest problems in deferring gratification, there is a neurological locus of these problems in the frontal cortex. Frontal lobe morphology, connectivity and functioning are still in the process of maturation during adolescence and young adulthood, and early exposure to substance use is another significant factor in the development of addiction. Many neuroscientists believe that developmental morphology is the basis that makes early-life exposure to substances such an important factor.

Genetic factors account for about half of the likelihood that an individual will develop addiction. Environmental factors interact with the person's biology and affect the extent to which genetic factors exert their influence. Resiliencies the individual acquires (through parenting or later life experiences) can affect the extent to which genetic predispositions lead to the behavioral and other manifestations of addiction. Culture also plays a role in how addiction becomes actualized in persons with biological vulnerabilities to the development of addiction.

Other factors that can contribute to the appearance of addiction, leading to its characteristic bio-psycho-socio-spiritual manifestations, include:
a. the presence of an underlying biological deficit in the function of reward circuits, such that drugs and behaviors which enhance reward function are preferred and sought as reinforcers;
b. the repeated engagement in drug use or other addictive behaviors, causing neuroadaptation in motivational circuitry leading to impaired control over further drug use or engagement in addictive behaviors;
c. cognitive and affective distortions, which impair perceptions and compromise the ability to deal with feelings, resulting in significant self-deception;
d. disruption of healthy social supports and problems in interpersonal relationships which impact the development or impact of resiliencies;
e. exposure to trauma or stressors that overwhelm an individual's coping abilities;
f. distortion in meaning, purpose and values that guide attitudes, thinking and behavior;
g. distortions in a person's connection with self, with others and with the transcendent (referred to as God by many, the Higher Power by 12-steps groups, or higher consciousness by others); and
h. the presence of co-occurring psychiatric disorders in persons who engage in substance use or other addictive behaviors.

Addiction is characterized by:
a. inability to consistently abstain;
b. impairment in behavioral control;
c. craving; or increased “hunger” for drugs or rewarding experiences;
d. diminished recognition of significant problems with one's behaviors and interpersonal relationships; and
e. a dysfunctional emotional response.

The power of external cues to trigger craving and drug use, as well as to increase the frequency of engagement in other potentially addictive behaviors, is also a characteristic of addiction, with the hippocampus being important in memory of previous euphoric or dysphoric experiences, and with the amygdala being important in having motivation concentrate on selecting behaviors associated with these past experiences.

Although some believe that the difference between those who have addiction, and those who do not, is the quantity or frequency of alcohol/drug use, engagement in addictive behaviors (such as gambling or spending), or exposure to other external rewards (such as food or sex), a characteristic aspect of addiction is the qualitative way in which the individual responds to such exposures, stressors and environmental cues. A particularly pathological aspect of the way that persons with addiction pursue substance use or external rewards is that preoccupation with, obsession with and/or pursuit of rewards (e.g., alcohol and other drug use) persist despite the accumulation of adverse consequences. These manifestations can occur compulsively or impulsively, as a reflection of impaired control.

Persistent risk and/or recurrence of relapse, after periods of abstinence, is another fundamental feature of addiction. This can be triggered by exposure to rewarding substances and behaviors, by exposure to environmental cues to use, and by exposure to emotional stressors that trigger heightened activity in brain stress circuits.

In addiction there is a significant impairment in executive functioning, which manifests in problems with perception, learning, impulse control, compulsivity, and judgment. People with addiction often manifest a lower readiness to change their dysfunctional behaviors despite mounting concerns expressed by significant others in their lives; and display an apparent lack of appreciation of the magnitude of cumulative problems and complications. The still developing frontal lobes of adolescents may both compound these deficits in executive functioning and predispose youngsters to engage in “high risk” behaviors, including engaging in alcohol or other drug use. The profound drive or craving to use substances or engage in apparently rewarding behaviors, which is seen in many patients with addiction, underscores the compulsive or avolitional aspect of this disease. This is the connection with “powerlessness” over addiction and “unmanageability” of life, as is described in Step 1 of 12 Steps programs.

Addiction is more than a behavioral disorder. Features of addiction include aspects of a person's behaviors, cognitions, emotions, and interactions with others, including a person's ability to relate to members of their family, to members of their community, to their own psychological state, and to things that transcend their daily experience.

Behavioral manifestations and complications of addiction, primarily due to impaired control, can include:
a. Excessive use and/or engagement in addictive behaviors, at higher frequencies and/or quantities than the person intended, often associated with a persistent desire for and unsuccessful attempts at behavioral control;
b. Excessive time lost in substance use or recovering from the effects of substance use and/or engagement in addictive behaviors, with significant adverse impact on social and occupational functioning (e.g. the development of interpersonal relationship problems or the neglect of responsibilities at home, school or work);
c. Continued use and/or engagement in addictive behaviors, despite the presence of persistent or recurrent physical or psychological problems which may have been caused or exacerbated by substance use and/or related addictive behaviors;
d. A narrowing of the behavioral repertoire focusing on rewards that are part of addiction; and
e. An apparent lack of ability and/or readiness to take consistent, ameliorative action despite recognition of problems.

Cognitive changes in addiction can include:
a. Preoccupation with substance use;
b. Altered evaluations of the relative benefits and detriments associated with drugs or rewarding behaviors; and
c. The inaccurate belief that problems experienced in one's life are attributable to other causes rather than being a predictable consequence of addiction.

Emotional changes in addiction can include:
a. Increased anxiety, dysphoria and emotional pain;
b. Increased sensitivity to stressors associated with the recruitment of brain stress systems, such that “things seem more stressful” as a result; and
c. Difficulty in identifying feelings, distinguishing between feelings and the bodily sensations of emotional arousal, and describing feelings to other people (sometimes referred to as alexithymia).

The emotional aspects of addiction are quite complex. Some persons use alcohol or other drugs or pathologically pursue other rewards because they are seeking “positive reinforcement” or the creation of a positive emotional state (“euphoria”). Others pursue substance use or other rewards because they have experienced relief from negative emotional states (“dysphoria”), which constitutes “negative reinforcement.” Beyond the initial experiences of reward and relief, there is a dysfunctional emotional state present in most cases of addiction that is associated with the persistence of engagement with addictive behaviors. The state of addiction is not the same as the state of intoxication. When anyone experiences mild intoxication through the use of alcohol or other drugs, or when one engages non-pathologically in potentially addictive behaviors such as gambling or eating, one may experience a “high”, felt as a “positive” emotional state associated with increased dopamine and opioid peptide activity in reward circuits. After such an experience, there is a neurochemical rebound, in which the reward function does not simply revert to baseline, but often drops below the original levels. This is usually not consciously perceptible by the individual and is not necessarily associated with functional impairments.

Over time, repeated experiences with substance use or addictive behaviors are not associated with ever increasing reward circuit activity and are not as subjectively rewarding. Once a person experiences withdrawal from drug use or comparable behaviors, there is an anxious, agitated, dysphoric and labile emotional experience, related to suboptimal reward and the recruitment of brain and hormonal stress systems, which is associated withdrawal from virtually all pharmacological classes of addictive drugs. While tolerance develops to the “high,” tolerance does not develop to the emotional “low” associated with the cycle of intoxication and withdrawal. Thus, in addiction, persons repeatedly attempt to create a “high”—but what they mostly experience is a deeper and deeper “low.” While anyone may “want” to get “high”, those with addiction feel a “need” to use the addictive substance or engage in the addictive behavior in order to try to resolve their dysphoric emotional state or their physiological symptoms of withdrawal. Persons with addiction compulsively use even though it may not make them feel good, in some cases long after the pursuit of “rewards” is not actually pleasurable. Although people from any culture may choose to “get high” from one or another activity, it is important to appreciate that addiction is not solely a function of choice. Simply put, addiction is not a desired condition.

As addiction is a chronic disease, periods of relapse, which may interrupt spans of remission, are a common feature of addiction. It is also important to recognize that return to drug use or pathological pursuit of rewards is not inevitable.

Clinical interventions can be quite effective in altering the course of addiction. Close monitoring of the behaviors of the individual and contingency management, sometimes including behavioral consequences for relapse behaviors, can contribute to positive clinical outcomes. Engagement in health promotion activities which promote personal responsibility and accountability, connection with others, and personal growth also contribute to recovery. It is important to recognize that addiction can cause disability or premature death, especially when left untreated or treated inadequately.

The qualitative ways in which the brain and behavior respond to drug exposure and engagement in addictive behaviors are different at later stages of addiction than in earlier stages, indicating progression, which may not be overtly apparent. As is the case with other chronic diseases, the condition must be monitored and managed over time to:
a. Decrease the frequency and intensity of relapses;
b. Sustain periods of remission; and
c. Optimize the person's level of functioning during periods of remission.

In some cases of addiction, medication management can improve treatment outcomes. In most cases of addiction, the integration of psychosocial rehabilitation and ongoing care with evidence-based pharmacological therapy provides the best results. Chronic disease management is important for minimization of episodes of relapse and their impact. Treatment of addiction saves lives.

Addiction professionals and persons in recovery know the hope that is found in recovery. Recovery is available even to persons who may not at first be able to perceive this hope, especially when the focus is on linking the health consequences to the disease of addiction. As in other health conditions, self-management, with mutual support, is very important in recovery from addiction. Peer support such as that found in various “self-help” activities is beneficial in optimizing health status and functional outcomes in recovery.

While there are many approaches to treatment no one has ever developed a novel test to determine outcome following treatment whether it involves just talk therapy, holistic modalities, neuro-genetic targeting, psychopharmacology, genomics and/or a combination of all of these worthy approaches. With this mind the inventors propose the first ever-test to determine outcome by tracking pre-and post mRNA gene expression as described herein.

The site of the brain where one experiences feelings of well being is the meso-limbic system. This part of the brain has been termed the “reward center”. The chemical messages include serotonin, enkephalins, GABA and dopamine, all working in concert to provide a net release of DA at the Nac (a region in the mesolimbic system). It is well known that genes control the synthesis, vesicular storage, metabolism, receptor formation and neurotransmitter catabolism. The polymorphic versions of these genes have certain variations that can lead to an impairment of the neurochemical events involved in the neuronal release of DA. The cascade of these neuronal events has been termed “Brain Reward Cascade”. A breakdown of this cascade will ultimately lead to a dysregulation and dysfunction of DA. Since DA has been established as the “pleasure molecule” and the “anti-stress molecule,” any reduction in function could lead to reward deficiency and resultant aberrant substance seeking behavior and a lack of wellness.

Homo sapiens physiology is motivationally programmed to drink, eat, have sex, and desire pleasurable experiences. Impairment in the mechanisms involved in these natural processes lead to multiple impulsive, compulsive and addictive behaviors governed by genetic polymorphic antecedents. While there are a plethora of genetic variations at the level of mesolimbic activity, polymorphisms of the serotonergic-2A receptor (5-HTT2a), dopamine D2 receptor (DRD2) and the Catechol-o-methyl-transferase (COMT) genes predispose individuals to excessive cravings and resultant aberrant behaviors.

An umbrella term to describe common genetic antecedents of multiple impulsive, compulsive and addictive behaviors is Reward Deficiency Syndrome (RDS). Individuals possessing a paucity of serotonergic and/or dopaminergic receptors and an increased rate of synaptic DA catabolism, due to high catabolic genotype of the COMT gene, are predisposed to self-medicating any substance or behavior that will activate DA release including alcohol, opiates, psychostimulants, nicotine, glucose, gambling, sex, and even excessive internet gaming, among others.

Acute utilization of these substances induces a feeling of well being. But, unfortunately, sustained and prolonged abuse leads to a toxic pseudo feeling of well being resulting in tolerance and disease or discomfort. Thus, low DA receptors due to carrying the DRD2 A1 allelic genotype results in excessive cravings and consequential behavior, whereas normal or high DA receptors results in low craving-induced behavior. In terms of preventing substance abuse, or excessive glucose craving, one goal would be to induce a proliferation of DA D2 receptors in genetically prone individuals. Experiments in vitro have shown that constant stimulation of the DA receptor system via a known D2 agonist results in significant proliferation of D2 receptors in spite of genetic antecedents. In essence, D2 receptor stimulation signals negative feedback mechanisms in the mesolimbic system to induce mRNA expression causing proliferation of D2 receptors. This molecular finding serves as the basis to naturally induce DA release to also cause the same induction of D2-directed mRNA and thus proliferation of D2 receptors in the human. This proliferation of D2 receptors in turn, will induce the attenuation of craving behavior. In fact this has been proven with work showing DNA-directed overexpression (a form of gene therapy) of the DRD2 receptors and significant reduction in both alcohol and cocaine craving-induced behavior in animals.

Finally, utilizing the long term dopaminergic activation approach will ultimately lead to a common safe and effective modality to treat RDS behaviors including Substance Use Disorders (SUD), Attention Deficit Hyperactivity Disorder (ADHD), and Obesity among other reward deficient aberrant behaviors. Support for the impulsive nature of individuals possessing dopaminergic gene variants is derived from a recent article suggesting that variants in the COMT gene predicts impulsive choice behavior and may shed light on treatment targets. The importance of neurochemical mechanisms involved in drug induced relapse behavior cannot be ignored. Using a drug relapse model, it has been shown previously that relapse can be induced by re-exposing rats to heroin-associated contexts, after extinction of drug-reinforced responding in different contexts, reinstates heroin seeking. This effect is attenuated by inhibition of glutamate transmission in the ventral tegmental area and medial accumbens shell, components of the mesolimbic dopamine system. This process enhances DA net release in the N. accumbens. This fits well with Li's KARG addiction network map.

### EXAMPLES

This section provides a number of examples whereby specific drugs and neuro-pathways interact in the genome to influence the biological function of mRNA as it relates to neurotransmission, enzymes involved in neurotransmitter metabolism as well as specific neuronal receptors common in producing a feeling of well-being in the animal or human.

In the basal ganglia, convergent input and dopaminergic modulation of the direct striatonigral and the indirect striatopallidal pathways are critical in rewarding and aversive learning and drug addiction. To explore how the basal ganglia information is processed and integrated through these two pathways, a reversible neurotransmission blocking technique was developed in which transmission of each pathway was selectively blocked by specific expression of transmission-blocking tetanus toxin in a doxycycline-dependent manner. The results indicated that the coordinated modulation of these two pathways was necessary for dopamine-mediated acute psychostimulant actions. This modulation, however, shifted to the predominant roles of the direct pathway in reward learning and cocaine sensitization and the indirect pathway in aversive behavior. These two pathways thus have distinct roles: the direct pathway critical for distinguishing associative rewarding stimuli from non-associative ones and the indirect pathway for rapid memory formation to avoid aversive stimuli. As for the role of drugs of abuse on mRNA involved in these pathways, thoughtful exploration, the following map has been developed, yielding for the first time a comprehensive set of gene-based biomarkers (e.g., mRNAs and/or the proteins encoded thereby) one, some, or all of which can be assayed utilizing, for example, array analysis to detect up- or down-regulation depending on the activity or substance (frequently a prescribed drug or drug of abuse) in question for a particular subject. (see Table 2, below).

### Example 1

### Utilizing GARS

In this test a Genetic Addiction Risk Score (GARS) is used to identify genes and related mRNA. See USSN 13/092,894, which is hereby incorporated by reference.

### Detailed Embodiment

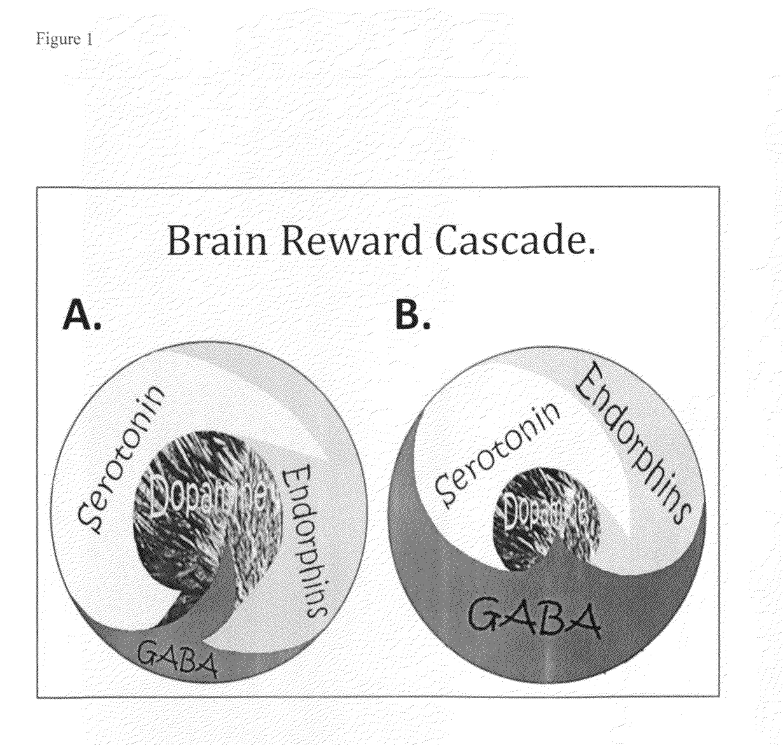

Over half a century of dedicated and rigorous scientific research on the meso-limbic system provided insight into the addictive brain and the neurogenetic mechanisms involved in man's quest for happiness. In brief, the site of the brain where one experiences feelings of well-being is the meso-limbic system. This part of the brain has been termed the “reward center”. Chemical messages including serotonin, enkephalins, GABA and dopamine (DA), work in concert to provide a net release of DA at the nucleous accumbens (NAc), a region in the mesolimbic system. It is well known that genes control the synthesis, vesicular storage, metabolism, receptor formation and neurotransmitter catabolism. The polymorphic-versions of these genes have certain variations that could lead to an impairment of the neurochemical events involved in the neuronal release of DA. The cascade of these neuronal events has been termed “Brain Reward Cascade” (see FIG. 1). A breakdown of this cascade will ultimately lead to a dysregulation and dysfunction of DA. Since DA has been established as the “pleasure molecule” and the “anti-stress molecule,” any reduction in function could lead to reward deficiency and resultant aberrant substance seeking behavior and a lack of wellness.

Homo sapiens are biologically predisposed to drink, eat, reproduce and desire pleasurable experiences. Impairment in the mechanisms involved in these natural processes lead to multiple impulsive, compulsive and addictive behaviors governed by genetic polymorphic antecedents. While there are a plethora of genetic variations at the level of mesolimbic activity, polymorphisms of the serotonergic-2A receptor (5-HTT2a); serotonergic transportor (5HTTLPR); (dopamine D2 receptor (DRD2), Dopamine D4 receptor (DRD4); Dopamine transporter (DAT1); and the Catechol-o-methyl-transferase (COMT), monoamine-oxidase (MOA) genes as well as other candidate genes predispose individuals to excessive cravings and resultant aberrant behaviors.

An umbrella term to describe the common genetic antecedents of multiple impulsive, compulsive and addictive behaviors is Reward Deficiency Syndrome (RDS). Individuals possessing a paucity of serotonergic and/or dopaminergic receptors and an increased rate of synaptic DA catabolism, due to high catabolic genotype of the COMT gene, or high MOA activity are predisposed to self-medicating with any substance or behavior that will activate DA release including alcohol, opiates, psychostimulants, nicotine, glucose, gambling, sex, and even excessive internet gaming, among others. Use of most drugs of abuse, including alcohol, is associated with release of dopamine in the mesocorticolimbic system or “reward pathway of the brain. Activation of this dopaminergic system induces feelings of reward and pleasure [6.7]. However, reduced activity of the dopamine system (hypodopaminergic functioning) can trigger drug-seeking behavior. Variant alleles can induce hypodopaminergic functioning through reduced dopamine receptor density, blunted response to dopamine, or enhanced dopamine catabolism in the reward pathway. Possibly, cessation of chronic drug use induces a hypodopaminergic state that prompts drug-seeking behavior in an attempt to address the withdrawal-induced state.

Acute utilization of these substances can induce a feeling of well being. But, unfortunately sustained and prolonged abuse leads to a toxic pseudo feeling of well being resulting in tolerance and disease or discomfort. Thus, low DA receptors due to carrying the DRD2 A1 allelic genotype results in excessive cravings and consequential behavior, Whereas normal or high DA receptors results in low craving induced behavior. In terms of preventing substance abuse, or excessive glucose craving, one goal would be to induce a proliferation of DA D2 receptors in genetically prone individuals. Experiments in vitro have shown that constant stimulation of the DA receptor system via a known D2 agonist in low doses results in significant proliferation of D2 receptors in spite of genetic antecedents. In essence, D2 receptor stimulation signals negative feedback mechanisms in the mesolimbic system to induce mRNA expression causing proliferation of D2 receptors. This molecular finding serves as the basis to naturally induce DA release to also cause the same induction of D2-directed mRNA and thus proliferation of D2 receptors in the human. This proliferation of D2 receptors in turn, will induce the attenuation of craving behavior. In fact this has been proven with work showing DNA-directed overexpression (a form of gene therapy) of the DRD2 receptors and significant reduction in both alcohol and cocaine craving-induced behavior in animals.

These observations are the basis for the development of a functional hypothesis of drug-seeking and drug use. The hypothesis is that the presence of a hypodopaminergic state, regardless of the source, is a primary cause of drug-seeking behavior. Thus, genetic polymorphisms that induce hypodopaminergic functioning may be the causal mechanism of a genetic predisposition to chronic drug use and relapse. Finally, utilizing the long term dopaminergic activation approach will ultimately lead to a common safe and effective modality to treat RDS behaviors including Substance Use Disorders (SUD), Attention Deficit Hyperactivity Disorder (ADHD), and Obesity among other reward deficient aberrant behaviors.

Support for the impulsive nature of individuals possessing dopaminergic gene variants is derived from a number of important studies illustrating the genetic risk for drug-seeking behaviors based on association and linkage studies implicating these alleles as risk antecedents having impact in the mesocorticolimbic system. The prime genes include but are not limited: least one of the RDS-associated alleles is an allele for a gene selected from the group consisting of DRD1, DRD2, DRD3, DRD4, DRD5, DAT1, PPARG, CHREBP, FTO, TNF-alpha, MANEA, Leptin OB, PEMT, MOAA, MOAB, CRH, CRHEP, CRHR1, CRHR2, GAL, NPY, NPY1R, NPY2R, NPYY5R, ADIPOQ, STS, VDR, DBI, 5HTTIRP, GABRA2, GABRA3, GABBRA4, GABRA5, GABRB1, GABRB2, GABRB3, GABRD, GABRE, GARG2, GABRG2, GABRG3, GARBQ, SLC6A7, SLC6A11, SLC6A13, SLC32A1, GAD1, GAD2, DB1, MTHFR, VEGF, NOS3, HTR3B, SLC6A3, SLC6A4, COMT, DDC, OPRD1, OPRM1, OPRK1, ANKK1, HTR2A, HTR2C, HTRIA, HTR1B, HTR2A, HTR2B, HTR2C, HTR3A, HTR3B, ALDH1, ALDH2, CAT, CYP2E1, ADH1A, ALDH1B, ALDH1C, ADH4, ADH5, ADH6, ADH7, TPH1, TPH2, CNR1, CYP2E1, OPRKI, PDYN, PNOC, PRD1, OPRL1, PENK, POMC, GLA1, GLRA1, GLRB, GPHN, FAAH, CHRM1, CHRM2, CHRM3, CHRM4, CHRM5, CHRNA4, CHRNB2, ADRA1A, ADRA2B, ADRB2, SLC6A2, DRA2A, DRA2C, ARRB2, DBH, SCL18A2, TH, GR1K1, GRIN1, GRIN2A, GRIN2B, GRIN2C, GRM1, SLC6A4, ADCY7, AVPR1A, AVPRIB, CDK5RI, CREB1, CSNKIE, FEV, FOS, FOSL1, FOSL2, GSKK3B, JUN, MAPK1, MAPK3, MAPK14, MPD2, MGFB, NTRK2, NTSRI, NTSR2, PPP1R1B, PRKCE, BDNF, CART, CCK, CCKAR, CCKBR, CLOCK, HCRT, LEP, OXT, NR3C1, SLC29A1, and TAC1.

The need to genetically test individuals especially at entry into a residential or even non-residential chemical dependency program has been suggested by scientists and clinicians alike here and abroad. In fact the most recent work of Conner et al. has suggested the importance of multiple hypodopaminergic gene polymorphisms as a possible predictive tool to identify children at risk for problematic drug use prior to the onset of drug dependence. A current exploratory study is in agreement with this prediction in terms of the development of a novel genetic test using an algorithm to determine the proposed GARS. To reiterate, it has been found that a high percentage (75%) of subjects carry a moderate to high GARS whereby 100% of individuals tested posses at least one risk allele tested.

### Preferred Embodiment for GARS Test

The hypodopaminergic state is likely due to gene polymorphisms as well as environmental elements including both stress and neurotoxicity from aberrant abuse of psychoactive drugs (i.e alcohol, heroin, cocaine etc). Genetic variables could include serotonergic genes (serotonergic receptors [5HT2a]; serotonin transporter 5HTIPR); endorphinergic genes (mu OPRM1 gene; proenkephalin (PENK) [PENK polymorphic 3′ UTR dinucleotide (CA) repeats}; GABergic gene (GABRB3) and dopaminergic genes (ANKKI Taq A; DRD2 C957T, DRD4 7R, COMT Val/met substation, MAO-A uVNTR, and SLC3 9 or 10R). Any of these genetic and or environmental impairments could result in reduced release of dopamine and or reduced number of dopaminergic receptors.

RDS GENE PANEL BASED ON META-ANALYSIS¹

| Gene | Significance | Comment |
|---|---|---|
| ALDH2** | P = 5 × 10⁻³⁷ | With alcoholism and alcohol- |
| | | induced medical diseases |
| ADH1B** | P = 2 × 10⁻²¹ | With alcoholism and alcohol- |
| | | induced medical diseases |
| ADH1C** | P = 4 × 10⁻³³ | With alcoholism and alcohol- |
| | | induced medical diseases |
| DRD2* | P = 1 × 10⁻⁸ | With alcohol and dug abuse |
| DRD4* | P = 1 × 10⁻² | With alcohol and drug abuse |
| SLC6A4 | P = 2 × 10⁻³ | With alcohol, heroin, cocaine, |
| | | methamphetamine dependence |
| HTRIB* | P = 5 × 10⁻¹ | With alcohol and drug abuse |
| HTRI2A* | P = 5 × 10⁻¹ | With alcohol and drug abuse |
| TPH* | P = 2 × 10⁻³ | With alcohol and drug abuse |
| MAOA* | P = 9 × 10⁻⁵ | With alcohol and drug abuse |
| OPRD1** | P = 5 × 10⁻¹ | With alcohol and drug abuse |
| GABRG2** | P = 5 × 10⁻⁴ | With alcohol and drug abuse |
| GABRA2* | P = 7 × 10⁻⁴ | With alcohol and drug abuse |
| GABRA6** | P = 6 × 10⁻⁴ | With alcohol and drug abuse |
| COMT* | P = 5 × 10⁻¹ | With alcohol and drug abuse in |
| | | Asians |
| DAT1* | P = 5 × 10⁻¹ | With alcohol and drug abuse in |
| | | Asians |
| CNR1* | P = 5 × 10⁻¹ | With alcohol and drug abuse |
| CYP2E1** | P = 7 × 10⁻² | With alcohol LIVER DISEASE |

Therefore utilizing GARS the mRNA outcome test for each patient follows the GARS diagnosis as they enter the treatment facility or primary care program.

TABLE 2
Substances/Activities of Choice
This table describes genes (and gene products, e.g., mRNA or protein) that can be analyzed in the context of the invention
with respect to various substances or activities of choice before and/or after ingestion or undertaking.

| Substance or | | | |
|---|---|---|---|
| Activity | mRNA increase | mRNA decrease | Citation(s) |
| high fat food (HFF) | 46% increase in TrkB in the VTA after 30 min of HFF | 38% decrease in BDNF in VTA after 60 min of HFF consumption [1] | [1] Cordeira, et al.; J Neurosci 2010 Feb 17; |
| | consumption [1] | Orexigenic Agrp downregulated 3-fold, NPY 0.57-fold in hypothalamus by | 30(7): 2533-41 |
| | Anorexigenic Cart upregulated 1.3-fold and Pomc 1.4-fold in | HFF [2] | [2] Lee, et al.; Nutrition 2010 Apr; 26(4): 411-22 |
| | hypothalamus [2] | Orexin receptor 2 in the hypothalamus [3] | [3] Tsuneki, et al.; Acta Physiol (Oxf) 2010 |
| | D2 receptor and/or the caudate putamen [4] | | Mar; 198(3): 335-48 |
| | D4 receptor in the ventromedial hypothalamic nucleus and ventral | | [4] Huang, et al.; Brain Res Mol Brain Res. |
| | part of lateral septal nucleus [4]. | | 2005 Apr 27; 135(1-2): 150-61 |
| nor-binaltorphimine | prodynorphin (PDYN) in NAc of DBA/2J and SWR/J mice (higher | PENK (lower in DBA/2J and SWR/J than in C57BL/6J) [1] | [1] Gieryk, et al.; Psychopharmacology (Berl) |
| (opioid receptor | than C57BL/6J) [1] | | 2010 Feb; 208(2): 291-300 |
| antagonist) | | | |
| housing and | | amygdala KOR and DOR opioid receptors; hypothalamic neuropeptide Y 5 | [1] Kalbe, et al.; Genes Brain Behav 2010 |
| cognitive | | receptor (NPY5R) [1] | Feb; 9(1): 75-83 |
| enrichment | | | |
| morphine | Mu receptors in mediobasal hypothalamus; | Mu receptors in NAc, caudate putamen (CPu), PAG; | [1] Le Merrer, et al.; Physiol Rev 2009 |
| | Kappa receptors in MBH; Penk in HPC, whole cortex, spinal cord; | Kappa receptors in NAc, striatum, PAG; Penk in NAc, CPu, HPT (PVN), | Oct; 89(4): 1379-412 |
| | Pdyn or Dyn in CPu and NAc [1] | FrCx, medulla oblongata (MO), nucleus paragigantocellularis; POMC in MBH | [3] Salas, et al.; Brain Res Bull. 2007 Jul |
| | Gpr88, Sgk, Cap1, PSD95, CamKII, DRD1A, Grm5, Adora2a, | and Arc, as well as HPT when withdrawal was precipated by naltrexone; | 12; 73(4-6): 325-9 |
| | Homer1, Cnr1, Gpr6 [2] | Pdyn or Dyn in CPu and NAc; Pdyn in HPC and HPT; Dyn in CPu and NAc | [4] Liu, et al.; Neuroscience 2005; 130(2): 282-8 |
| | hsp90beta [3] | [1] | [5] Romualdi, et al.; Neuroreport 2002 Apr |
| | ProorphaninFQ/N in nucleus accumbens, temporo-parietal cortex | CryB, CCK, Aq4, Gpr123, Gpr5, Gal [2] | 16; 13(5): 645-8 |
| | and striatum area in response to single injection 10 mg/kg. Chronic | Chronic administration caused decrease of proorphaninFQ/N in striatum and | |
| | administration caused significant increase in ventral tegmental area | nucleus accumbens [5] | |
| | [5] | | |
| morphine | Mu receptors in NAc, CPu, LH; Penk in striatum and HPT; | Penk in CPu, NAc, pons, spinal cord; depending on how withdrawal was | [1]Le Merrer, et al.; Physiol Rev 2009 |
| withdrawal | POMC in pituitary [1] | induced (spontaneously or by injecting an opioid antagonist), a decrease or | Oct; 89(4): 1379-412 |
| | orexin in lateral hypothalamus of Fischer 344 inbred rats (w/ no | no change in Penk expression measured in rostral PAG [1] | [2] Zhou, et al.; 2008 Neuroscience |
| | change in ppDyn) [2] | Alpha-synuclein in mouse basolateral amygdala, dorsal striatum, nucleus | [4] Bice, et al.; Mamm Genome 2008 |
| | POMC in anterior pituitary, mu opioid receptor in lateral | accumbens, and ventral tegmental area [6] | Feb; 19(2): 69-76 |
| | hypothalamus, nucleus accumbens core, and caudate-putamen; | | [5] Zhou, et al.; J Endocrinol. 2006 |
| | orexin in lateral hypothalamus [5] | | Oct; 191(1): 137-45 |
| | | | [6] Ziolkowska, et al.; J Neurosci. 2005 May |
| | | | 18; 25(20): 4996-5003 |
| ethanol | Mu receptors in inferior colliculus; Penk expression in PVN; | Mu receptors in HPT in both alcohol preferring and non-preferring following | [1] Le Merrer, et al.; Physiol Rev 2009 |
| | POMC in MBH after 3 weeks of gradual removal of ethanol; | chronic ethanol; Kappa receptors in VTA and NAc following chronic ethanol; | Oct; 89(4): 1379-412 |
| | Pdyn in HPC; Pdyn in CPu, Tu, and NAc in response to | Penk in striatum, Pir, and Tu. Penk expression decreased in VMH; POMC in | [2] Kuzmin, et al.; Brain Res 2009 Dec 11; 1305 |
| | ethanol withdrawal [1] proenkephalin in caudate-putamen [3] | MBH; Pdyn in HPT, hippocampus [1] | [3] Mendez, et al.; J Mol Neurosci 2008 |
| | cAMP-PKA signaling in prefrontal cortex, lateral and medial | pronociceptin (PNOC), 1.7-fold in hippocampus of alcoholics | Mar; 34(3): 225-34 |
| | septum, basolateral amygdala, paraventricular and anterior | opiate receptor-like 1 (OPRL-1) 1.4-fold in amygdala of alcoholics [2] | [4] Vadasz, et al.; Genomics 2007 |
| | hypothalamus, centromedial thalamus, CA1 region of hippocampus | proenkephalin in substantia nigra pars compacta and pars reticulata [3] | Dec; 90(6_: 690-72 |
| | and dentate gyrus, substantia nigra pars compacta, ventral | Drd2 in nucleus accumbens and hippocampus [4] | [5] Asyyed, et al.; Brain Res. 2006 Aug |
| | tegmental area, geniculate nucleus and superior colliculus [5] | Pro-opiomelanocortin mRNA expression of beta-endorphin neurons in the | 23; 1106(1): 63-71 |
| | CART in nucleus accumbens (effect blocked by both SCH-23390 | arcuate nucleus of rats [7] | [6] Salinas, et al.; J Neurochem 2006 |
| | and raclopride pretreatment) [6] | All 8 GABA receptor subunits, 4 of 5 subunits of different glutamate | Apr; 97(2): 408-15 |
| | PENK in nucleus accumbens 1 h after onset of intragastric infusion | receptors, and 7 enzymes involved with GABA and glutamate production | [7] Checn, et al.; J Neurochem 2004 |
| | [9] | (GAD-65, GAD-67, glutaminase, glutamate dehydrogenase, glutamine | Mar; 88(6): 1547-54 |
| | | synthetase, aspartate aminotransferase (cytosolic and mitochondrial), | [8] Eravci, et al.; Br J Pharmacol. 2000 |
| | | cytochrome oxidase subunit III, VIc, ATP synthase subunits A and C, Na K | Oct; 131(3)423-32 |
| | | ATPase subunit alpha 1and beta 1)) were reduced almost exclusively in the | [9] Li, et al.; Brain Res 1998 May 25; 794(1): |
| | | parieto-occipital cortex [8] | 35-47 |
| cocaine | Mu receptors in NAc and rostral cingulate cortex; Increased Penk | Decreased kappa receptor expression in NAc and VTA when cocaine | [1] Le Merrer, et al.; Physiol Rev 2009 |
| | in CPu, NAc*; Pdyn in CPu, dentate gyrus of HPC [1] | administered alone or in combination with ethanol; Kappa receptors | Oct; 89(4): 1379-412 |
| | micro-RNA miR-181a in mesolimbic dopaminergic system [2] | decreased in SN under chronic binge cocaine (but not after withdrawal); | [2] Chandrasekar et al 2009 |
| | NRXN3 beta expression in the globus pallidus [4] | hypothalamic Pdyn [1] | [3] Zhou et al 2008 Neuroscience |
| | CART in sublenticular extended amygdala [5] | micro-RNAs miR-124, let-7d in dopaminergic reward system, leading to | [4] Kelai, et al.; Neuroreport 2008 May |
| | Chronic cocaine upregulates En1 [6]. | downregulation of BDNF and D3R [2] | 7; 19(7): 751-5 |
| | CD81 (tetraspanin transmembrane protein involved in cell | orexin after cocaine place conditioning in lateral hypothalamus of Sprague- | [5] Fagergren, et al.; hysiol Behav 2007 Sep |
| | adhesion) in nucleus accumbens following acute cocaine treatment. | Dawley rats [3] | 10; 92(1-2): 218-25 |
| | [8] | Chronic cocaine downregulates Nurr1 and Pitx3 [6]. | [6]Riva, et al.; Exp Neural 2007 |
| | Dynorphin in medial caudate putamen [9] | Prodynorphin in animals with perinatal drug exposure [10] | Feb; 203(2): 472-80 |
| | CART in the amygdala [10] | Tyrosine hydroxylase in midbrain [12] | [7] Hall, et al.; Neuropsychopharmacology. |
| | D3 receptor in nucleus accumbens increased 6-fold in cocaine | | 2003 Aug; 28(8): 1485-90 |
| | overdose victims [11] | | [8] Brenz, et al.; Mol Cell Neurosci 2001 |
| | Dopamine receptors; preprodynorphin and preproenkephalin; | | Feb; 17(2): 303-16 |
| | dynorphin in striatum, enkephalin in both frontal cortex and striatal | | [9] Werme, et al.; Eur J Neurosci 2000 |
| | areas [12] | | Aug; 12(8): 2067-74 |
| | | | [10] Hurd, et al.; Ann N Y Aced Sci 1999 Jun |
| | | | 29; 877: 499-506 |
| | | | [11] Segal, et al.; Brain Res Mol Brain Res |
| | | | 1997 May; 45(2): 335-9 |
| | | | [12] Chai, et al.; J Neurosci 1997 Feb |
| | | | 1; 17(3): 1112-21 |
| cocaine withdrawal | Mu receptors in frontal cortex; Pdyn in CPu [1] | Penk in CPu and NAc, VMN, CeA; Pdyn in CPu [1] | [1] Le Merrer, et al.; Physiol Rev 2009 |
| | orexin and ppDyn in the lateral hypothalamus [2] | | Oct; 89(4): 1379-412 |
| | | | [2] Zhou et al 2008 Neuroscience |
| Amphetamine | Penk in frontal cortex; Pdyn in AMG [1] | Penk in CeA and anterior medial CPu [1] | [1] Le Merrer, et al.; Physiol Rev 2009 |
| | mGluR8 in rat dorsal and ventral striatum, as well as cortex, inc. | GluR1 in nucleus accumbens shell, GluR2 in core and shell [3]. | Oct; 89(4): 1379-412 |
| | cingulate and sensory but not piriform cortex (increase sustained | | [2] Parelkar, et al.; Neurosci Lett. 2008 Mar |
| | up to 21 days of withdrawal) [2] | | 15; 433(3): 250-4 |
| | After 3 days of withdrawal, GluR1 in PFC [3] | | [3] Lu, et al.; Synapse 1999 May; 32(2): 119-31 |
| amphetamine | Mu receptors in VTA; Pdyn in CPu and NAc [1] | | [1] Le Merrer, et al.; Physiol Rev 2009 |
| withdrawal | | | Oct; 89(4): 1379-412 |
| Chronic nicotine | Mu receptors in VTA; POMC in Arc; POMC in AL of the | POMC in MBH which was observed after 21 days of spontaneous withdrawal | [1] Le Merrer, et al.; Physiol Rev 2009 |
| treatment | pituitary; Pdyn in CPu after nicotine withdrawal; Pdyn in HPT [1] | from nicotine; Pdyn in ventral shell of NAc [1] | Oct; 89(4): 1379-412 |
| | c-fos in bed nucleus of stria terminalis, nucleus accumbens shell | Dopamine D2 receptor and tyrosine hydroxylase in PC12 clonal cell line from | [2] Naha et al 2009 |
| | and VTA. c-fos in central amygdala, locus coeruleus, nucleus | chromaffin adrenal cells [2] | [3] Shram, et al.; Neurosci Lett. 2007 May |
| | accumbens, paraventricular nucleus of hypothalamus, and lateral | | 18; 418(3): 286-91 |
| | septum [3]. | | [4] Walters, et al.; Neuron 2005 Jun |
| | CREB phosphorylation when exposed to situation where previous | | 16; 46(6): 933-43 |
| | nicotine reward was experienced [4] | | [5] Leslie, et al.; Ann N Y Aced Sci. 2004 |
| | MOR expression [4] | | Jun; 1021: 148-59 |
| | c fos in limbic regions of adolescents [5] | | |
| Alcohol cessation | delta receptor transcripts in striatum of alcohol-avoiders[1] | | [1] Le Merrer, et al.; Physiol Rev 2009 |
| | | | Oct; 89(4): 1379-412 |
| Cannabinoid | Increased Penk in NAc and CPu, Tu and Pir, HPT (both PVN and | | [1] Le Merrer, et al.; Physiol Rev 2009 |
| agonists (THC, | VMH), mammillary area and PAG; Increased POMC in Arc, | | Oct; 89(4): 1379-412 |
| CP-55,940 or | lasting up to 14 days following cessation [1] | | |
| R-methanandamide) | | | |
| cannabinoid | Penk in CPu, NAc, Tu, Pir. [1] | | [1] Le Merrer, et al.; Physiol Rev 2009 |
| withdrawal | | | Oct; 89(4): 1379-412 |
| Kappa receptor | Pdyn in HPT [1] | Pdyn in CPu, HPC, FrCx HPT [1] | [1] Le Merrer, et al.; Physiol Rev 2009 |
| agonists (U-69593 | | | Oct; 89(4): 1379-412 |
| or U-50,488H) | | | |
| Methamphetamine | Pdyn in HPT [1] | Animals that are TNF-alpha (−/−) have attenuated meth-induced increases in | [1] Le Merrer, et al.; Physiol Rev 2009 |
| | Increased TNF-alpha in normal animals [2] | extracellular striatal DA [2] | Oct; 89(4): 1379-412 |
| | | | [2] Nakajima, et al.; J Neurosci. 2004 Mar |
| | | | 3; 24(9): 2212-25 |
| food (effects on | Deprivation upregulated FTO [1] | | [1] Olszewski, et al.; BMC Neurosci 2009 Oct |
| hypothalamic FTO) | | | 27; 10: 129 |
| Leucine | | FTO in hypothalamus of rodents [1] | [1] Olszewski, et al.; BMC Neurosci 2009 Oct |
| | | | 27; 10: 129 |
| dual orexin receptor | Inhibits ability of subchronic amphetamine to produce behavioral | | [1] Winrow, et al.; Neuropharmacology 2010 |
| antagonist | sensitization and blocks alteration of gene expression levels in | | Jan; 58(1): 185-94 |
| (DORA) - | response to amphetamine exposure (particularly those associated | | |
| antagonist of OX1R | with synaptic plasticity in the VTA). DORA attenuates the | | |
| and OX2R | ability of nicotine to induce reinstatement of extinguished | | |
| | responding for reinforcer [1] | | |
| Aging | | orexin-receptor 2 mRNA in hypothalamus [1] | [1] Tsuneki, et al.; Acta Physiol (Oxf) 2010 |
| | | | Mar; 198(3): 335-48 |
| CREB | mCREB (a dominant-negative CREB which acts as a CREB | Overexpression of mutant CREB leads to a decrease in dynorphin | [1] DiNieri, et al.; J Neurosci 2009 Feb |
| | antagonist) animals are more sensitive to rewarding effects of | transcription [2] | 11; 29(6): 1855-9 |
| | cocaine, and insensitive to depressive-like effects of kappa | Blockade of kappa oioid receptors (on which dynorphin acts) antagonizes the | [2] Carlezon, et al.; Science 1998 Dec |
| | opioid receptor agonist U50,488 [1] | negative effect of CREB on cocaine reward [2] | 18282(5397): 2272-5 |
| | Overexpression CREB in mice leads to increased dynorphin | | |
| | transcription [2] | | |
| dopamine | DAT overexpressing rats showed increased impulsivity and risk | | [1] Adriani, et al.; Neuroscience 2009 Mar |
| transporter (DAT - | proneness—thus reduced dopaminergic tone following altered | | 3; 159(1): 47-58 |
| as influenced by | accumbal DAT function subserve a sensation-seeker phenotype | | |
| overexpression or | and vulnerability of impulse-control disorders [1] | | |
| silencing in the | | | |
| nucleus accumbens) | | | |
| CREB | CART in the nucleus accumbens [1] | | [1]Rogge, et al.; Brain Res 2009 Jan |
| | | | 28; 1251: 42-52 |
| deoxyribozyme 164 | | [1] ERK and CREB in frontal cortex, hippocampus, and striatum | [1] Li, et al.; Am J Drug Alcohol Abuse 2008; |
| (DRz164)- cleaves | | | 34(6): 673-82 |
| Period 1 gene | | | |
| (Per1) mRNA. | | | |
| Injection with | | | |
| DRz164 before | | | |
| morphine treatment | | | |
| para- | [1] repeated stress in pre-treated animals led to less glucocorticoid | [1] repeated stress in pre-treated rats led to downregulation of BDNF mRNA | [1] Zhou, et al.; Behav Brain Res 2008 Dec |
| chloroamphetamine | receptor increase | | 16; 195(1): 129-38 |
| (depletes 5-HT) | | | |
| predisposition for | G-alpha q—endogenous negative regulator of VMAT2 [1] | tyrosine hydroxylase, VMAT2, DAT, D2S presynaptic autoreceptor [1] | [1] Geiger, et al.; FASB J. 2008 |
| obesity (normal | | | Aug; 22(8): 2740-6 |
| diet) | | | |
| editing of serotonin | 5HT-2C expression and editing in the Nucleus Accumbens shell | | [1] Dracheva, et al.; |
| 2C receptor mRNA | compared with PC and VTA—also in general editing is higher in | | Neuropsychopharmacology. 2009 |
| (via ADAR | rats with a locomotor high response [1] | | Sep; 34(10): 2237-51 |
| enzyme) | | | |
| Heroin | PENK polymorphic 3′UTR dinucleotide (CA) repeats common in | DAT in paranigral nucleus and mesolimbic division of the ventral tegmental | [1] Nikoshkov, et al.; Proc Natl Acad Sci USA |
| | heroin abuse. Express higher PENK mRNA [1] | area. Reduction of Nurr1 expression with age in heroin users [2] | 2008 Jan 15; 105(2): 786-91 |
| | TH and alpha synuclein in VTA PN in heroin users with no change | tyrosine hydroxylase in mesolimbic dopamine neurons [3] | [2] Horvath, et al.; J Neurosci. 2007 Dec |
| | in the D2 receptor [2] | | 5; 27(49): 13371-5 |
| | PENK; NAc PENK in Met/Met (control) heroin abusers [3] | | [3] Nikoshkov, et al.; Proc Natl Acad Sci USA |
| | | | 2008 Jan 15; 105(2): 786-91 |
| social isolation | | dopamine D2 receptors in Flinders rats [1] | [1] Bjornebekk, et al.; Neuroreport 2007 Jul |
| | | | 2; 18(10): 1039-43 |
| HSV vector | Elevated GluR1 transcription when delivered GluR1 by vector [1] | Vector-mediated elevated GluR2 leads to decreases in prodynorphin [1] | [1] Todtenkopf, et al.; J Neurosci 2006 Nov |
| mediated elevations | | | 8; 26(45): 11665-9 |
| in GluR1 or | | | |
| GluR2 | | | |
| high or low | Differences in expression of 5HT2A, mGlu1 in hippocampus, and | Differences in expression of 5HT2A, mGlu1 in hippocampus, and AMPA | [1] Pickering, et al.; Neurobiol Learn Mem. |
| consumption of | AMPA GluR1 and adrenergic alpha 2A in PFC. NMDA NR2B, | GluR1 and adrenergic alpha 2A in PFC. NMDA NR2B, GABA Alpha 3 in | 2007 Feb; 87(2): 181-91 |
| sugar | GABA Alpha 3 in PFC and adrenergic alpha2B and alpha2A, | PFC and adrenergic alpha2B and alpha2A, AMPA, GluR1, GluR2, GluR3, | |
| | AMPA, GluR1, GluR2, GluR3, 5HT1B and GABA alpha 5 in | 5HT1B and GABA alpha 5 in hippocampus [1] | |
| | hippocampus [1] | | |
| Leptin receptor | Leptin activates intracellular JAK-STAT pathway and reduction in | Direct administration of leptin to VTA caused decreased food intake while | [1] Hommel, et al.; Neuron 2006 Sep |
| expression in VTA | firing rate [1] | long-term RNAi mediated knockdown of Lep in VTA led to increased food | 21; 51(6): 801-10 |
| | | intake [1] | |
| ethanol preference | Gsta4 (glutathione-S-transferase alpha 4) [1] | decreased fatty acid amidohydrolase (FAAH) expression in PFC of alcohol | [1] Bjork, et al.; FASEB J 2006 |
| | | preferring animals, accompanied by decreased binding of CB1 receptor | Sep; 20(11): 1826-35 |
| | | ligand (3)[H]SR141716A and [35S]GTPgammaS incorporation stimulated by | [2] Hansson, et al.; Neuropsychopharmacology |
| | | the CB1 agonist WIN 55, 212-2. This suggests an overactive | 2007 Jan; 32(1): 117-26 |
| | | endocannabinoid transmission in PFC of alcohol preferring animals and | |
| | | compensatory downregulation of CB1 signaling. [2] | |
| morphine response | Differences in opiate response with corresponding differences in | Differences in opiate response with corresponding differences in Atp I aw, | [1] Korostynski, et al.; BMC Genomics 2006 |
| (mice) | Atp I aw, COMT, Gabra I, GABA-A, Gabra2, Grm7, Kcnj 9, Syt4, | COMT, Gabra I, GABA-A, Gabra2, Grm7, Kcnj 9, Syt4, Gfap, Mtap2, and | Jun 13; 7: 146 |
| | Gfap, Mtap2, and Hprt I [1] | Hprt I [1] | |
| psychostimulant | CART in ventral tegmental area, nucleus accumbens [1] | | [1] Jaworski, et al.; Peptides 2006 |
| (e.g. cocaine, | Modulation of CART peptides by psychostimulants may involve | | Aug; 27(8): 1993-2004 |
| amphetamine) | corticosterone and/or cAMP response element binding protein | | |
| | (CREB) [1] | | |
| forskolin (intra- | CART—effect attenuated by inhibition of PKA with H89 [N-(2-[p- | | [1] Jones, et al.; J Pharmacol Exp Ther. 2006 |
| accumba l injection | bromocinnamylamino]ethyl)-5-isoquinoline-sulfonamide | | Apr; 317(1): 454-61 |
| in rat) | hydrochloride and adenosine-3′,5′ cyclinc monophosphorothioate, | | |
| | Rp-isomer, OR Rp-cAMPS alone. [1] | | |
| intrastriatal infusion | | striatal enkephalin gene expression, an effect that greatly suppresses food | [1] Kelley, et al.; J Comp Neurol 2005 Dec 5; |
| of cholinergic | | intake [1] | 493(1): 72-85 |
| muscarinic | | | |
| antagonist | | | |
| Delta-tetrahydro- | BDNF in reward center (nucleus accumbens, medial prefrontal | — | Butovsky, et al.; J Neurochem 2005 |
| cannabinol | cortex and paraventricular nucleus) [1] | | May; 93(4): 802-11 |
| | zif268, blocked by SL327 an inhibitor of MAPK/ERK kinase, | | [2] Valjent, et al.; Eur J Neurosci. 2001 |
| | as well as SCH 2339 [2] | | Jul; 14(2): 342-52 |
| | THC induces a progressive and transient activation | | |
| | (phosphorylation) of MAPK/ERK in dorsal striatum and nucleus | | |
| | accumbens. This activation is totally inhibited by selective | | |
| | antagonist of CBD cannabinoid receptors, SR 141716A. [2] | | |
| DeltaFosB | prolonged DeltaFosB expression increased drug reward [1] | | [1] McClung, et al.; Nat Neurosci 2003 |
| | | | Nov; 6(11): 1208-15 |
| Nandrolone | Dopamine D(2) receptor at the lowest doses in the caudate | Dopamine D(1)-receptor subtype in the caudeate putamen and nucleus | Kindlundh, et al.; Brain Res. 2003 Jul 25; |
| decanoate | putamen and nucleus accumbens [1] | accumbens shell (at higher doses) [1] | 979(1-2): 37-42 |
| Voluntary wheel | | | |
| running in addicted | | | |
| Lewis rats | | | |
| Substance P (during | | D2 receptor in nucleus accumbens and frontal cortex [1] | Zhou, et al.; Peptides 2003 Jan; 24(1): 147-53 |
| morphine | | | |
| withdrawal) | | | |
| U99194A (D(3) | c-fos (similar pattern to that produced by d-amphetamine) in | | Carr, et al.; Psychopharmacology (Berl) 2002 |
| dopamine receptor | caudate-putamen and nucleus accumbens, blocked by SCH-23390 | | Aug; 163(1): 76-84 |
| antagonist) | [1] | | |
| cocaine, cocaine + | | cocaine alone or cocaine and nandrolone caused decrease in NR1 in the | [1] Le Greves, et al.; Acta Psychiatr Scand |
| nondrolone, or | | nucleus accumbens. Combined treatment significantly down-regulated the | Suppl 2002; (412): 129-32 |
| nandrolone alone | | transcript in the periaqueductal gray compared with other groups. [1] | |
| Dextromethorphan | 40 mg/kg ip in rats caused increase of tyrosine hydroxylase (TH) | | [1] Zhang, et al.; Neurosci Lett. 2001 Aug |
| | mRNA in VTA and substantia nigra [1] | | 24; 309(2): 85-8 |
| Running | dynorphin in medial caudate putamen [1] | AMPA receptor [2] | [1] Werme, et al.; Eur J Neurosci ′00 |
| | GluR1 in ventral tegmentum [2] | NGFI-B and Nor1 in cerebral cortex [3] | Aug; 12(8): 2967-74 |
| | | | [1] Makatsori, et al.; |
| | | | Psychoneuroendocrinology 2003 Jul; 28(5): |
| | | | 702-14 |
| | | | [3] Werme, et al.; J Neurosci 1999 Jul |
| | | | 15; 19(14): 6169-74 |
| Amitriptyline | dopamine D3 receptor mRNA in shell of the nucleus accumbens; | | [1] Lammers, et al.; Mol Psychiatry 2000 |
| | D1 and D2 receptors [1] | | Jul; 5(4): 378-88 |
| Desipramine | dopamine D3 receptor mRNA in shell of the nucleus accumbens | | [1] Lammers, et al.; Mol Psychiatry 2000 |
| | [1] | | Jul; 5(4): 378-88 |
| Imipramine | dopamine D3 receptor mRNA in shell of the nucleus accumbens; | | [1] Lammers, et al.; Mol Psychiatry 2000 |
| | D1 and D2 receptors [1] | | Jul; 5(4): 378-88 |
| Tranylcypromine | dopamine D3 receptor mRNA in shell of the nucleus accumbens | | [1] Lammers, et al.; Mol Psychiatry 2000 |
| | [1] | | Jul; 5(4): 378-88 |
| electroconvulsive | 10 days of treatment led to increased dopamine D3 receptor mRNA | | [1] Lammers, et al.; Mol Psychiatry 2000 |
| therapy | in shell of the nucleus accumbens [1] | | Jul; 5(4): 378-88 |
| Fetal alcohol | c-fos, c-jun, jun B, and zif268 in prefrontal cortex, hippocampal | junB in caudate nucleus [1] | [1] Nagahara, et al.; Alcohol Clin Exp Res 1995 |
| syndrome | subfields CA1 and CA3 [1] | | Dec; 19(6): 1389-97 |
| S(−)- and R (+)- | | POMC anterior pituitary cell line [1] | [1] Putscher, et al.; Alcohol 1995 Sep-Oct; |
| salsolinol | | Decrease in cAMP level occurs after treatment with S(−)-SAL, whereas R(+)- | 12(5): 447-52 |
| | | SAL does not affect cAMP production [1] | |
| peripheral nerve | tyrosine hydroxylase and DRD2 in nucleus accumbens (changes in | | [1] Austin, et al.; Neuroscience 2010 Nov |
| injury (unilateral | DRD2 expression were not observed with disability (only with pain | | 24; 171(1): 329-43 |
| chronic constriction | resulting from injury)) [1] | | |
| of sciatic nerve) | | | |
| Alcohol and splice | D2L/D2S receptor ratio in the pituitary gland; ethanol consumption | | [1] Sasabe, et al.; Int J Environ Res Public |
| variants | may increase NMDA NR1 isoforms that are weakly inhibited by | | Health 2010 Apr; 7(4): 1448-66 |
| | ethanol [1] | | |

### Example 3

### Commonality Test

TABLE 3
Common RDS gene expression of mRNA
(based on drug of choice effects)

| mRNA up | mRNA down |
|---|---|
| TrkB | Orexigenic Agrp |
| Pomc | NPY |
| D4 | Orexin receptor 2 |
| prodynorphin (PDYN) | KOR |
| Mu receptors | DOR |
| Kappa receptors | neuropeptide Y 5 receptor (NPY5R) |
| Dyn | Gal |
| Gpr88 | CryB |
| Sgk | Aq4 |
| Cap1 | Gpr123 |
| PSD95, | Gpr5 |
| CamKII | opiate receptor-like 1 (OPRL-1) |
| DRD1A | All 8 GABA receptor subunits |
| Grm5 | glutamate receptors |
| Adora2a, | ERK |
| Homer1 | Na K ATPase subunit alpha 1 and |
| | beta 1 |
| Cnr1 | GAD-65 |
| Gpr6 [ | GAD-67 |
| hsp90beta | Glutaminase |
| ProorphaninFQ/N | glutamate dehydrogenase |
| Orexin | glutamine synthetase |
| cAMP-PKA | aspartate aminotransferase |
| CART | cytochrome oxidase subunit III |
| micro-RNA miR-181a | VIc, |
| NRXN3 beta | ATP synthase subunits A and C |
| En1 | Nurr1 |
| D3 receptor | Pitx3 |
| Preproenkephalin | VMAT2 |
| mGluR8 | fatty acid amidohydrolase (FAAH) |
| GluR1 | AMPA receptor |
| MOR | CB1 |
| CREB phosphorylation | NR1 |
| c fos | Nor1 |
| delta receptor | NGFI-B |
| FTO | ANK11-kinase (Ala239) |
| glucocorticoid receptor | Neurotensin |
| G-alpha q-endogenous negative | |
| regulator of VMAT2 | |
| 5HT-2C | |
| TH | |
| alpha synuclein | |
| intracellular JAK-STAT | |
| Gsta4 (glutathione-S-transferase | |
| alpha 4) | |
| BDNF i | |
| DeltaFosB | |
| Dopamine D(2) receptor | |
| tyrosine hydroxylase | |
| alpha 6 subunit in | |
| catecholaminergic | |
| nuclei | |
| c-jun | |
| jun B, | |
| zif268 | |
| CCK | |
| Neurotensin | |
| dopamine reuptake transporter | |
| COMT | |
| MAO-A, | |
| Slc12a6 | |
| Dlgap2 | |
| Etnk1 | |
| Palm | |
| Sqstm1 | |
| Nsg1 | |
| Akap9 | |
| Apba1 | |
| Stau1 | |
| Elavl4 | |
| Kif5a | |
| Syt1 | |
| Hipk2 | |
| Araf, | |
| Cmip | |
| NMDA | |
| NR1 | |

Methods for Detecting mRNA

This invention involves the collection of any cell-containing tissue (e.g., blood, skin, saliva, a buccal swab, hair, etc.) for extraction of mRNA or protein by any appropriate method.

### Whole-Genome Gene Expression Profiling

In one embodiment, a strategy of detailed time-course studies of gene expression alterations following pre- and post entry to residential and or non-residential treatment using Illumina Whole-Genome 6 microarrays. To analyze the dynamics of early, intermediate and relatively late changes in mRNA abundance, the analysis will be performed at different time points for example: upon entry; two weeks, 4 weeks and during recovery.

Support for this methodology is based on microarray data analysis using two-way ANOVA identified 42 drug-responsive genes with P<1×10⁻⁶ (corresponding to P<0.05 after adjusting for approximately 48,000 independent tests using Bonferroni correction). Compared to other gene expression profiling studies, the statistical threshold was rather conservative. However, the same threshold is widely accepted in population genetic and genome-wide association studies in humans. The difference between the methodological standards may result from the number of samples and biological replicates usually used in these two types of whole-genome studies.

In one study, the maximum number of true positive genes altered in the striatum by drugs of abuse (drug factor, 104 transcripts) was found at a 29% FDR. Beyond that level, the number of true positives did not increase. Surprisingly, the number of true positives remained stable (84 to 104 transcripts, mean=94.4±4.9) over a wide range of FDR (4.7 to 56.3%). The results for the drug factor are in contrast to alterations in the striatal gene expression profile related to the time point of the experiment (time factor). The maximum number of true positive genes (5,442 transcripts) for the time factor was found at a 69.8% FDR and increased linearly in the range 0.1 to 69.8% FDR. The above observations suggest a rather unexpected conclusion. While the diurnal cycle alters a vast fraction of the brain transcriptome, drugs regulated the expression of a limited number of genes (approximately 100), and this alteration was robust. The number of genes obtained using Bonferroni correction (42 transcripts) was equal to the number of genes obtained at a 0.1% FDR threshold. Therefore, at the chosen threshold, we identified 40.3% (42 of 104 transcripts) of genes altered by drugs of abuse with 99.9% confidence.

The changes in mRNA abundance of selected marker genes were validated by quantitative PCR (qPCR) using aliquots of the non-pooled total RNA. (yielding an overall correlation between the microarray and qPCR results of r=0.69 (Spearman's method, P=4.87×10⁻²⁴). The alterations in mRNA level were also confirmed in an independent experiment. In addition, the expression of the selected genes was evaluated during the acquisition and expression of morphine-induced CPP.

Correlation with Behavioral Drug Effects

To link the gene expression patterns with drug-related phenotypes, others have analyzed the correlations between the transcriptional and behavioral drug effects in mice. Mutual interactions between the brain gene expression and behavioral profiles are complex and multidimensional. Therefore, it is difficult to define them using analyses performed with only the few available data points. However, even speculative results obtained from this analysis create the unique possibility of assigning different transcriptional alterations induced by various drugs to drug-related phenotypes. A positive correlation of r=0.62 (Pearson's method, P<0.001) was observed between the level of drug-induced locomotor activation and the degree of transcriptional response of gene expression pattern A. Additionally, a significant correlation between the acute induction of Bi genes and the rewarding effect of the drug (r=0.7, Pearson's method, P<0.05, was found. This provides confidence that gene expression induced by various drugs are linked to expected behaviors, including RDS behaviors.

Evaluation of Two Drug-Regulated Genes at the mRNA and Protein Levels

Western blotting has been used to determine whether the changes in gene expression are translated into alterations in protein levels. As such, the morphine-induced increase in Sgk1 abundance as been associated with a significant decrease in the level of the protein (0.75-fold). Therefore, Sgk1 expression changes might be a compensatory effect to the loss of the protein. Up-regulation of Tsc22d3 has been associated with an increase in the corresponding protein level (approximately 1.5-fold). Double-immuno-fluorescence labeling with neuronal (NeuN) and astroglial (S100B) markers have been used to identify cells that expressed SGK (Sgk1) and GILZ (Tsc22d3) proteins. In the mouse striatum, both genes appeared to be expressed mainly in neurons.

The above methodology is presented as an example of how it is feasible to develop assays for the relationship between drugs of abuse and behavioral effects that will lead to a test to determine treatment outcome.

### CONCLUSION

The genetic tests described herein are important for understanding treatment response in any given RDS scenario. While it is not specific for any drug of abuse in terms of treatment program it will be useful for providing important info regarding not only drug abuse treatment but programs involved in treatment of food cravings and obesity. In one example, along with the potential solution involving the formulation KB220Z, information related to an understanding of why this complex could affect mRNA expression in a number of well-known pathways. See U.S. Pat. No. 6,955,873.

An overwhelming segment of the world's population possesses certain genetic variations that increase risk for genetic predispositions that preclude them from reaching their optimum health potential, contribute to impaired health, and/or can cause involuntary indulgence in detrimental and self-destructive behaviors. This is especially true for products that empower individuals to successfully overcome compulsions and excessive cravings, like those that lead to unwanted and unhealthy weight gain, and other health woes that burden society (i.e. addictions, depression, and other related problems).

It is believed that the genesis of all behavior, whether so-called normal (socially acceptable) or abnormal (socially unacceptable) behavior, derives from an individual's genetic makeup at birth. This predisposition, due to multiple gene combinations and polymorphisms, is expressed differently based on numerous environmental elements. It is further believed that the core of predisposition to these behaviors is a set of genes, which promote a feeling of well-being via neurotransmitter interaction at the “reward site” of the brain (located in the meso-limbic system), leading to normal dopamine release and influencing dopamine receptor density. The DRD2 gene is responsible for the synthesis of dopamine D2 receptors. And, further depending on the genotype (allelic form A1 versus A2), the DRD2 gene dictates the number of these receptors at post-junctional sites.

A low number of D2 receptors suggest a hypodopaminergic function as manifested in addictive disorders. When there are a low number of dopamine receptors, the person will be more prone to seek any substance or behavior that stimulates the dopaminergic system (a sort of “dopamine fix”). To understand generalized craving behavior, due to hypodopaminergic function, individuals self-medicate through biochemical (illicit or non-illicit) attempts to alleviate or compensate for the low dopaminergic brain activity via drug-receptor activation (alcohol, heroin, cocaine, glucose, etc.). This will substitute for the lack of reward and yield a temporary compensatory sense of well-being. In order to help explain this so called pseudo self-healing process, it is germane that the reinforcing properties of many drugs of abuse may be mediated through activation of common neurochemical pathways, particularly with regard to the meso-limbic dopamine system and as such these drugs will have profound influence on gene expression thereof.

In predisposed genotypes, gene polymorphic expression (and resulting aberrant behavior) is amplified in response to chronic nutritional deficiencies from habitual dietary patterns that are chronically unable to meet the greater nutrient needs mandated by those polymorphisms manifesting as RDS. In this regard, glucose, opiates, nicotine, cocaine, tetrahydrocannibinol (THC), and ethanol (among others) have been shown to directly or indirectly enhance release or block re-uptake of dopamine. These findings suggest the importance of genotyping polymorphisms of the dopaminergic and other reward pathways to develop a ‘genetic positioning system’ map (GPS). To date, there are numerous clinical trials showing various recovery benefits from RDS behaviors using KB220.

The results of these studies support an interaction of KB220 and meso-limbic activation leading to “normalization” of abnormal dopaminergic function in anticipation of patients carrying a number of reward gene polymorphisms. It appears that KB220 is the only natural “Dopamine Agonist” without any negative side-effects that are common among pharmaceutical medications. In fact, KB220 has been able to demonstrate that it was able increase the positive effects of alpha and low beta activity in the Parietal regions of the brain compared to placebo. The fact that KB220 induced an increase in both alpha and low beta activity seems to mimic the protocol used in neurofeedback to treat alcoholics. This indicates that KB220 “normalizes” brain abnormalities associated with drug dependency (alcohol, heroin and psycho stimulants) induced because of dopaminergic deficiency by acting as a Dopaminergic receptor agonist during extended abstinence in polydrug abusers.

Clinicians are interested in the potential of increasing the number of DR2R that long-term activation of dopaminergic receptors (i.e., DRD2 receptors) by KB220 should accomplish. This phenomena will lead to enhanced “dopamine sensitivity”, greater self-control, and an increased sense of happiness. However, to date there is no outcome measure that definitively enables real objective assessment of patients in terms of outcome. Using the concept of treating RDS victims with KB220Z, as one example of treatment, the methods herein provide novel information for the first time ever.

Thus, the coupling of these methods as way to display the actual role of treatment will provide a descriptive gene expression and/or protein map.

All patents, patent applications, and publications mentioned in the specification are indicative of the levels of those of ordinary skill in the art to which the invention pertains. Each patent, patent application, and publication cited herein is hereby incorporated by reference in its entirety for all purposes regardless of whether it is specifically indicated to be incorporated by reference in the particular citation.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

I claim:

1. A method of objective assessment of a Reward Deficiency Syndrome (RDS) behavior in a subject known to have or suspected of having RDS, wherein the method comprises obtaining a first expression profile on a biological sample obtained from the subject at a first time point and a second expression profile on a biological sample obtained from the subject at a second time point, wherein the first and second expression profiles comprise measuring a level of an expression product, optionally a messenger RNA (mRNA) or a protein, for at least one gene selected from the group consisting of TrkB, Pomc, D4, prodynorphin (PDYN), Mu receptors, Kappa receptors, Dyn, Gpr88, Sgk, Cap1, PSD95, CamKII, DRD1A, Grm5, Adora2a, Homer1, Cnr1, Gpr6, hsp90beta, ProorphaninFQ/N, Orexin, cAMP-PKA, CART, micro-RNA miR-181a, NRXN3 beta, Ent, D3 receptor, Preproenkephalin, mGluR8, GluR1, MOR, CREB phosphorylation, c fos, delta receptor, FTO, glucocorticoid receptor, G-alpha q-endogenous negative regulator of VMAT2, 5HT-2C, TH, alpha synuclein, intracellular JAK-STAT, Gsta4 (glutathione-S-transferase alpha 4), BDNF I, DeltaFosB, Dopamine D(2) receptor, tyrosine hydroxylase, alpha 6 subunit in catecholaminergic nuclei, c-jun, jun B, zif268, CCK, Neurotensin, dopamine reuptake transporter, COMT, MAO-A, Slc12a6, Dlgap2, Etnk1, Palm, Sqstm1, Nsg1, Akap9, Apba1, Stau1, Elavl4, Kif5a, Syt1, Hipk2, Araf, Cmip, NMDA, and NR1.

2. A method according to claim 1 wherein the first expression profile is conducted prior to delivering a therapy to the subject intended to treat or alter the course of the Reward Deficiency Syndrome (RDS) behavior.

3. A method according to claim 2 that further comprises:
a. performing an allelic analysis on a biological sample from the subject to determine if the subject's genome contains at least one RDS-associated allele for each of two genes selected from the group consisting of DRD1, DRD2, DRD3, DRD4, DRD5, DAT1, PPARG, CHREBP, FTO, TNF-alpha, MANEA, Leptin OB, PEMT, MOAA, MOAB, CRH, CRHEP, CRHR1, CRHR2, GAL, NPY, NPY1R, NPY2R, NPYY5R, ADIPOQ, STS, VDR, DBI, 5HTTIRP, GABRA2, GABRA3, GABBRA4, GABRA5, GABRB1, GABRB2, GABRB3, GABRD, GABRE, GARG2, GABRG2, GABRG3, GARBQ, SLC6A7, SLC6A11, SLC6A13, SLC32A1, GAD1, GAD2, DB1, MTHFR, VEGF, NOS3, HTR3B, SLC6A3, SLC6A4, COMT, DDC, OPRD1, OPRM1, OPRK1, ANKK1, HTR2A, HTR2C, HTRIA, HTR1B, HTR2A, HTR2B, HTR2C, HTR3A, HTR3B, ALDH1, ALDH2, CAT, CYP2E1, ADH1A, ALDH1B, ALDH1C, ADH4, ADH5, ADH6, ADH7, TPH1, TPH2, CNR1, CYP2E1, OPRKI, PDYN, PNOC, PRD1, OPRL1, PENK, POMC, GLA1, GLRA1, GLRB, GPHN, FAAH, CHRM1, CHRM2, CHRM3, CHRM4, CHRM5, CHRNA4, CHRNB2, ADRA1A, ADRA2B, ADRB2, SLC6A2, DRA2A, DRA2C, ARRB2, DBH, SCL18A2, TH, GR1K1, GRIN1, GRIN2A, GRIN2B, GRIN2C, GRM1, SLC6A4, ADCY7, AVPR1A, AVPRIB, CDK5RI, CREB1, CSNKIE, FEV, FOS, FOSL1, FOSL2, GSKK3B, JUN, MAPK1, MAPK3, MAPK14, MPD2, MGFB, NTRK2, NTSRI, NTSR2, PPP1R1B, PRKCE, BDNF, CART, CCK, CCKAR, CCKBR, CLOCK, HCRT, LEP, OXT, NR3C1, SLC29A1, and TAC1, wherein the allelic analysis is performed before, concurrently, or after the first expression profile; and, optionally,
b. determining a genetic addiction risk based on the results of the allelic analysis, wherein the genetic addiction risk takes into the account the presence of one or more of RDS-associated alleles among the genes analyzed, wherein the presence of at least one RDS-associated allele indicates a genetic addiction risk.

4. A method according to claim 2 wherein the second expression profile is conducted after delivering a therapy to the subject intended to treat or alter the course of the Reward Deficiency Syndrome (RDS) behavior.

5. A method according to claim 1 wherein the biological samples are derived from tissue samples obtained from the subject, wherein optionally the tissue samples are cell-containing samples optionally selected from the group consisting of blood, hair, mucous, saliva, and skin.

6. A method according to claim 1 wherein one or more of the expression profiles is a gene expression profile or a protein expression profile.

7. A method according to claim 1 wherein one or more of the expression profiles is a gene expression profile obtained from a messenger RNA-containing biological sample or a protein expression profile obtained from a protein-containing biological sample.

8. A method according to claim 1 wherein the RDS behavior is the subject's self-administration of a substance or activity of choice, wherein optionally the substance or activity of choice is selected from the group consisting of:
ddd. high fat food (HFF), wherein optionally the first and second expression profile experiments assess the mRNA of at least one gene selected from the group consisting of TrkB, Cart, Pomc, D2 receptor, D4 receptor, BDNF, Agrp, NPY, and Orexin receptor 2;
eee. nor-binaltorphimine (opioid receptor antagonist), wherein optionally the first and second expression profile experiments assess the mRNA of at least one gene selected from the group consisting of PDYN and PENK;
fff. housing and cognitive enrichment, wherein optionally the first and second expression profile experiments assess the mRNA of at least one gene selected from the group consisting of amygdala KOR and DOR opioid receptors and NPY5R;
ggg. morphine, wherein optionally the first and second expression profile experiments assess the mRNA of at least one gene selected from the group consisting of Mu receptors, Kappa receptors, PENK, PDYN, DYN, Gpr88, Sgk, Cap1, PSD95, CamKII, DRD1A, Grm5, Adora2a, Homer1, Cnr1, Gpr6, hsp90beta, ProorphaninFQ/N, POMC, CryB, CCK, Aq4, Gpr123, Gpr5 and Gal;
hhh. morphine withdrawal, wherein optionally the first and second expression profile experiments assess the mRNA of at least one gene selected from the group consisting of Mu receptors, POMC, orexin, PENK and Alpha-synuclein;
iii. ethanol, wherein optionally the first and second expression profile experiments assess the mRNA of at least one gene selected from the group consisting of Mu receptors, PENK, POMC, PDYN, cAMP-PKA, CART, PNOC, OPRL-1, Drd2, all 8 GABA receptor subunits, 4 of 5 subunits of different glutamate receptors, and 7 enzymes involved with GABA and glutamate production (GAD-65, GAD-67, glutaminase, glutamate dehydrogenase, glutamine synthetase, aspartate aminotransferase (cytosolic and mitochondrial), cytochrome oxidase subunit III, Vlc, ATP synthase subunits A and C, Na K ATPase subunit alpha 1 and beta 1));
jjj. cocaine, wherein optionally the first and second expression profile experiments assess the mRNA of at least one gene selected from the group consisting of Mu receptors, PENK, PDYN, micro-RNA miR-181a, NRXN3 beta expression, CART, Ent, CD81, D3 receptor, Depamine receptors, ppDYN, DYN, Kappa Receptors, micro-RNAs miR-124, BDNF, D3R, orexin, Nurr1, Pitx3 and tyrosine hydroxylase;
kkk. cocaine withdrawal, wherein optionally the first and second expression profile experiments assess the mRNA of at least one gene selected from the group consisting of Mu receptors, PDYN, orexin, ppDYN and PENK;
lll. Amphetamine, wherein optionally the first and second expression profile experiments assess the mRNA of at least one gene selected from the group consisting of PENK, PDYN, mGluR8, GluR1 and GluR2;
mmm. amphetamine withdrawal, wherein optionally the first and second expression profile experiments assess the mRNA of at least one gene selected from the group consisting of Mu receptors and PDYN;
nnn. Chronic nicotine treatment, wherein optionally the first and second expression profile experiments assess the mRNA of at least one gene selected from the group consisting of Mu receptors, POMC, PDYN, c-Fos, CREB phosphorylation, dopamine D2 receptor and tyrosine hydroxylase;
ooo. Alcohol cessation, wherein optionally the first and second expression profile experiments assess the mRNA of at least one gene selected from the group consisting of delta receptor;
ppp. Cannabinoid agonists (THC, CP-55,940 or R-methanandamide), wherein optionally the first and second expression profile experiments assess the mRNA of at least one gene selected from the group consisting of PENK and POMC;
qqq. cannabinoid withdrawal, wherein optionally the first and second expression profile experiments assess the mRNA of at least one gene selected from the group consisting of PENK;
rrr. Kappa receptor agonists (U-69593 or U-50,488H), wherein optionally the first and second expression profile experiments assess the mRNA of at least one gene selected from the group consisting of PDYN;
sss. Methamphetamine, wherein optionally the first and second expression profile experiments assess the mRNA of at least one gene selected from the group consisting of PDYN and TNF-alpha;
ttt. food (effects on hypothalamic FTO), wherein optionally the first and second expression profile experiments assess the mRNA of at least one gene selected from the group consisting of FTO;
uuu. Leucine, wherein optionally the first and second expression profile experiments assess the mRNA of at least one gene selected from the group consisting of FTO;
vvv. dual orexin receptor antagonist (DORA)-antagonist of OX1R and OX2R, wherein optionally the first and second expression profile experiments assess the mRNA of at least one gene selected from the group consisting of
www. Aging, wherein optionally the first and second expression profile experiments assess the mRNA of at least one gene selected from the group consisting of orexin-receptor 2;
xxx. CREB, wherein optionally the first and second expression profile experiments assess the mRNA of at least one gene selected from the group consisting of
yyy. dopamine transporter (DAT—as influenced by overexpression or silencing in the nucleus accumbens), wherein optionally the first and second expression profile experiments assess the mRNA of at least one gene selected from the group consisting of
zzz. CREB, wherein optionally the first and second expression profile experiments assess the mRNA of at least one gene selected from the group consisting of CART;
aaaa. deoxyribozyme 164 (DRz164)—cleaves Period 1 gene (Peri) mRNA. Injection with DRz164 before morphine treatment, wherein optionally the first and second expression profile experiments assess the mRNA of at least one gene selected from the group consisting of ERK and CREB;
bbbb. para-chloroamphetamine (depletes 5-HT), wherein optionally the first and second expression profile experiments assess the mRNA of at least one gene selected from the group consisting of glucocorticoid receptor and BDNF;
cccc. predisposition for obesity (normal diet), wherein optionally the first and second expression profile experiments assess the mRNA of at least one gene selected from the group consisting of Galphaq, tyrosine hydroxylase, VMAT2, DAT, and D2S presynaptic autoreceptor;
dddd. editing of serotonin 2C receptor mRNA (via ADAR enzyme), wherein optionally the first and second expression profile experiments assess the mRNA of at least one gene selected from the group consisting of 5HT-2C;
eeee. Heroin, wherein optionally the first and second expression profile experiments assess the mRNA of at least one gene selected from the group consisting of PENK, D2 receptor, DAT, Nurr1 and tyrosine hydroxylase;
ffff. social isolation, wherein optionally the first and second expression profile experiments assess the mRNA of at least one gene selected from the group consisting of D2 receptor;
gggg. HSV vector mediated elevations in GluR1 or GluR2, wherein optionally the first and second expression profile experiments assess the mRNA of at least one gene selected from the group consisting of GluR1 and GluR2;
hhhh. high or low consumption of sugar, wherein optionally the first and second expression profile experiments assess the mRNA of at least one gene selected from the group consisting of 5HT2A, mGlu1, AMPA, GluR1, adrenergic alpha 2A, NMDA NR2B, GABA Alpha 3, adrenergic alpha2B, GluR2, GluR3, 5HT1B and GABA alpha5;
iiii. Leptin receptor expression in VTA, wherein optionally the first and second expression profile experiments assess the mRNA of at least one gene selected from the group consisting of
jjjj. ethanol preference, wherein optionally the first and second expression profile experiments assess the mRNA of at least one gene selected from the group consisting of Gsta4, FAAH and CB1;
kkkk. morphine response (mice), wherein optionally the first and second expression profile experiments assess the mRNA of at least one gene selected from the group consisting of Atp I aw, COMT, Gabra I, GABA-A, Gabra2, Grm7, Kcnj 9, Syt4, Gfap, Mtap2, and Hprt I;
iiii. psychostimulant (e.g. cocaine, amphetamine), wherein optionally the first and second expression profile experiments assess the mRNA of at least one gene selected from the group consisting of CART, cAMP and CREB;
mmmm. forskolin (intra-accumbal injection in rat), wherein optionally the first and second expression profile experiments assess the mRNA of at least one gene selected from the group consisting of CART;
nnnn. intrastriatal infusion of cholinergic muscarinic antagonist, wherein optionally the first and second expression profile experiments assess the mRNA of at least one gene selected from the group consisting of
oooo. Delta-tetrahydrocannabinol, wherein optionally the first and second expression profile experiments assess the mRNA of at least one gene selected from the group consisting of BDNF, zif268 and MAPK/ERK;
pppp. DeltaFosB, wherein optionally the first and second expression profile experiments assess the mRNA of at least one gene selected from the group consisting of
qqqq. Nandrolone decanoate, wherein optionally the first and second expression profile experiments assess the mRNA of at least one gene selected from the group consisting of D2 receptor and D1 receptor;
rrrr. Voluntary wheel running in addicted Lewis rats, wherein optionally the first and second expression profile experiments assess the mRNA of at least one gene selected from the group consisting of
ssss. Substance P (during morphine withdrawal), wherein optionally the first and second expression profile experiments assess the mRNA of at least one gene selected from the group consisting of D2 receptor;
tttt. U99194A (D(3) dopamine receptor antagonist), wherein optionally the first and second expression profile experiments assess the mRNA of at least one gene selected from the group consisting of c-Fos;
uuuu. cocaine, cocaine+nondrolone, or nandrolone alone, wherein optionally the first and second expression profile experiments assess the mRNA of at least one gene selected from the group consisting of
vvvv. Dextromethorphan, wherein optionally the first and second expression profile experiments assess the mRNA of at least one gene selected from the group consisting of tyrosine hydroxylase;
wwww. Running, wherein optionally the first and second expression profile experiments assess the mRNA of at least one gene selected from the group consisting of DYN, GluR1, AMPA, NGFI-B and Nor1;
xxxx. Amitriptyline, wherein optionally the first and second expression profile experiments assess the mRNA of at least one gene selected from the group consisting of D1, D2 and D3 receptors;
yyyy. Desipramine, wherein optionally the first and second expression profile experiments assess the mRNA of at least one gene selected from the group consisting of D3 receptor;
zzzz. Imipramine, wherein optionally the first and second expression profile experiments assess the mRNA of at least one gene selected from the group consisting of D1, D2 and D3 receptors;
aaaaa. Tranylcypromine, wherein optionally the first and second expression profile experiments assess the mRNA of at least one gene selected from the group consisting of D3 receptor;
bbbbb. electroconvulsive therapy, wherein optionally the first and second expression profile experiments assess the mRNA of at least one gene selected from the group consisting of D3 receptor;
ccccc. Fetal alcohol syndrome, wherein optionally the first and second expression profile experiments assess the mRNA of at least one gene selected from the group consisting of c-fos, c-jun, jun B, zif268 and junB;
ddddd. S(−)- and R (+)-salsolinol, wherein optionally the first and second expression profile experiments assess the mRNA of at least one gene selected from the group consisting of POMC and cAMP;
eeeee. peripheral nerve injury (unilateral chronic constriction of sciatic nerve), wherein optionally the first and second expression profile experiments assess the mRNA of at least one gene selected from the group consisting of tyrosine hydroxylase and DRD2; and
fffff. alcohol and splice variants, wherein optionally the first and second expression profile experiments assess the mRNA of at least one gene selected from the group consisting of D2UD2S receptor ratio and NMDA NR1.
